(19) United States
(12) Patent Application Publication
(10) Pub. No.: US 2010/0317593 A1
(43) Pub. Date: Dec. 16, 2010

(54) 2,3-DIHYDRO-1H-INDENE COMPOUNDS

(75) Inventors: Brian Aquila, Marlborough, MA (US); Edward Hennessy, Roslindale, MA (US); Alexander Hird, Waltham, MA (US); Vibha Oza, Acton, MA (US); Jamal Carlos Saeh, Watertown, MA (US); Li Sha, Wincester, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/796,089

(22) Filed: Jun. 8, 2010

Related U.S. Application Data
(60) Provisional application No. 61/186,594, filed on Jun. 12, 2009.

(51) Int. Cl. A61K 38/06; C07K 7/02; A61P 35/00; A61P 35/02

(52) U.S. Cl. 514/19.4; 514/19.3; 514/19.5; 514/19.6; 514/21.9; 530/323

(57) ABSTRACT

Provided herein are 2,3-dihydro-1H-indene compounds, methods for making the compounds, pharmaceutical compositions containing the compounds. The described compounds inhibit IAP proteins and can be used to treat various cancers.

### PRIORITY

The present application claim priority to U.S. provisional patent application 61/186,594, filed on Jun. 12, 2009, the entire contents of which are hereby incorporated by reference.

### FIELD

Provided herein are 2,3-dihydro-1H-indene compounds, pharmaceutical compositions containing these compounds and methods for their preparation. The described compounds inhibit IAP proteins and can be used to treat various cancers.

### BACKGROUND

Inhibitor of apoptosis proteins (IAPs) were initially identified in baculoviruses, where they play a role in replication by preventing infected cells from undergoing apoptosis. Subsequently, multiple IAPs have been found in insects, nematodes and vertebrates where some members play an important role in development while others are involved in cell cycle control. Two motifs present in the baculovirus IAP protein have been identified in cellular IAPs. The baculovirus IAP repeat (BIR) domain is approximately 70-80 amino acids long and contains a Zn-binding motif. The presence of a BIR domain is what defines a protein as a member of the IAP family. BIR domains facilitate protein-protein interactions involved in IAP function. A second motif found in the baculovirus IAP and some cellular IAPs is the really interesting new gene (RING) finger, a type of Zn-finger found in other proteins, which in the IAPs has E3-ubiquitin ligase activity. The human genome contains eight IAPs: cIAP1, cIAP2, XIAP, Ts-IAP, Livin, survivin, NAIP and Apollon or Bruce. (Hunter, A. M., E. C. LaCasse and R. G. Korneluk, 2007, The inhibitors of apoptosis (IAPs) as cancer targets, Apoptosis, 12:1543-1568.)

XIAP has three BIR domains (BIR1, 2 and 3) and a RING finger. It can directly inhibit apoptosis through its ability to bind to the active form of several members of the caspase family of proapoptotic proteases. The XIAP BIR3 domain binds to the N-terminus of activated caspase-9 preventing caspase-9 dimer formation, which is essential for activity. Caspases-3 and -7 bind to the linker region between the BIR1 and 2 domains blocking the caspase active site. (Riedl, S. J. and Y Shi, 2004, Molecular Mechanisms of Caspase Regulation During Apoptosis, Nat. Rev. Mol. Cell. Biol., 5: 897-907)

cIAP1 and cIAP2 were initially identified by interaction with the type 2 tumor necrosis factor-α receptor complex [TNFR2] (Rothe, M. et al. 1995, The TNFR2-TRAF Signaling Complex Contains Two Novel Proteins Related to Baculoviral Inhibitor of Apoptosis Proteins, Cell, 83: 1243-1252). Both cIAP1 and cIAP2 contain three BIR domains (BIR1, 2 and 3), a RING finger and a caspase recruitment domain (CARD). cIAP1 binds to TRAF1/2 in the TNFR2 complex through its BIR1 domain (Samuel, T., K. Welsh, T. Lober, S. H. Togo, J. M. Zapata and J. C. Reed, 2006, Distinct BIR Domains of cIAP1 Mediate Binding to and Ubiquitination of Tumor Necrosis Factor Receptor-associated Factor 2 and Second Mitochondrial Activator of Caspases, J. Biol. Chem. 281: 1080-1090). In the activated TNFR complex ubiquitination of RIPK1 by the RING domain of cIAP1 and cIAP2 is a key step in activating TAK1 signaling downstream of the TNFR and leads to activation of the prosurvival, canonical NF-kappaB pathway and synthesis of the caspase-8 inhibitor FLIP (Varfolomeev E., et al. 2008 c-IAP1 and c-IAP2 are Critical Mediators of Tumor Necrosis Factor α (TNFα)-induced NF-kappaB Activation, J. Biol. Chem., 283: 24295-24299.). cIAP1 also acts to negatively regulate the non-canonical NF-kappaB pathway by ubiquitination and subsequent proteosomal degradation of NIK. Like XIAP, cIAP1 and cIAP2 can bind to caspases in vitro, however, the affinity by which they bind does not appear to be physiologically relevant (Eckelman, B. P. and G. S. Salvesen, 2006, The Human Anti-apoptotic Proteins cIAP1 and cIAP2 Bind but Do Not Inhibit Caspases, J. Biol. Chem. 281: 3254-3260.).

A cellular antagonist of the BIR2 and 3 domains of XIAP, cIAP1 and cIAP2, along with the single BIR domain of Livin, is called the second mitochondrial activator of caspases (SMAC). SMAC is synthesized in the cytoplasm and then imported into the mitochondria where its N-terminal 55 amino acids are cleaved from the rest of the protein. Upon loss of mitochondrial integrity, as can occur upon DNA damage or treatment with agents that lead to apoptosis, mitochondrial SMAC enters the cytoplasm where it binds to XIAP, cIAP1, cIAP2 and Livin. SMAC binding to these IAPs is facilitated by binding of the N-terminal 4 amino acids, AVPI, to the BIR2 and/or 3 domains of cIAP1, cIAP2, XIAP and the single BIR domain of ML-IAP (Hunter et al.)

SMAC binding to XIAP prevents XIAP from inhibiting caspases-3, -7 and -9 and thus is proapoptotic. SMAC binding to cIAP1 and cIAP2 leads to autoubiquitination and proteosome-mediated degradation of cIAP1 and cIAP2. Loss of cIAP1 and cIAP2 inhibits signaling downstream of the TNFR through the canonical NF-kappaB pathway. In cells in which an active complex of TNF-alpha and TNFR occurs, it also leads to caspase-8 activation through the formation of a complex between TRAD, RIPK1 and procaspase-8. The formation of active caspase-8 and the absence of FLIP, due to inactivation of the canonical NF-kappaB pathway, leads to apoptosis in these tumor cells.

Structural studies have shown that the XIAP BIR3 domain binds to the N-terminal 4 amino acids of SMAC, Ala-Val-Pro-Ile (AVPI). Biochemical studies have shown that AVPI and related peptides also bind to cIAP1, cIAP2 and Livin BIR domains. Cell permeable, small molecule mimetics of AVPI (SMAC mimetics) that bind to XIAP, ML-IAP, cIAP1 and cIAP2 have been made. However, there remains a need for novel compounds that target one or more IAPs, which exploit these apoptosis-linked pathways.

### SUMMARY

Provided herein are compounds of formula I:

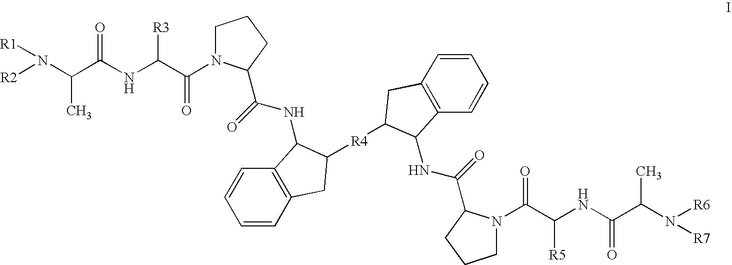

In the compounds of Formula I:

R1 and R2 are independently H or C(1-6)alkyl;
R3 is H or C(3-8)cycloalkyl;
R4 is —OC(3-10)alkylO—, —OC(3-10)alkenylO— or —OC(3-10)alkynylO—,
R5 is H or C(3-8)cycloalkyl; and
R6 and R7 are independently H or C(1-6)alkyl.

Forms of the compounds can include salts, such as pharmaceutically acceptable salts, solvates or hydrates of the described compounds. The described compounds can also be part of a pharmaceutical composition, which can additionally include a pharmaceutically acceptable carrier, diluent or excipient.

The compounds and compositions inhibit IAP activity and can be used accordingly, such as in the treatment of cancer. Accordingly, the compounds and compositions can be used as a medicament. For example, the compounds may be used to treat acute myeloid leukemia, bladder cancer, breast cancer, colon cancer, diffuse large B-cell lymphoma, non-small cell lung cancer, ovarian cancer, pancreatic cancer, or prostate cancer.

### DETAILED DESCRIPTION

Provided herein are compounds of formula I.

In some compounds, one of R1 and R2 is a C(1-6)alkyl and the other of R1 and R2 is H. In some compounds, one of R1 and R2 is a methyl and the other of R1 and R2 is H. In additional compounds both R1 and R2 are H.

In some compounds, R3 is C(3-8)cycloalkyl, for example cyclohexyl.

In embodiments of these and other compounds, R4 is

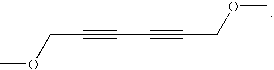

In other embodiments, R4 is

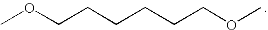

In these and other embodiments, R5 is C(3-8)cycloalkyl, for example cyclohexyl.

In still other embodiments, one of R6 and R7 is a C(1-6)alkyl and the other of R6 and R7 is H. In yet further embodiments, one of R6 and R7 is a methyl and the other of R6 and R7 is H. In other embodiments both R6 and R7 are H.

In some embodiments, one of R1 and R2 is a C(1-6)alkyl, the other of R1 and R2 is H, R3 is C(3-8)cycloalkyl, R4 is —OC(3-10)alkynylO—, R5 is C(3-8)cycloalkyl, one of R6 and R7 is a C(1-6)alkyl, and the other of R6 and R7 is H.

In other embodiments, one of R1 and R2 is a methyl, the other of R1 and R2 is H, R3 is cyclohexyl, R4 is

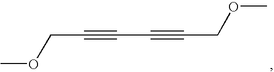

R5 is cyclohexyl, one of R6 and R7 is a methyl, and the other of R6 and R7 is H.

Specific compounds of formula I are:

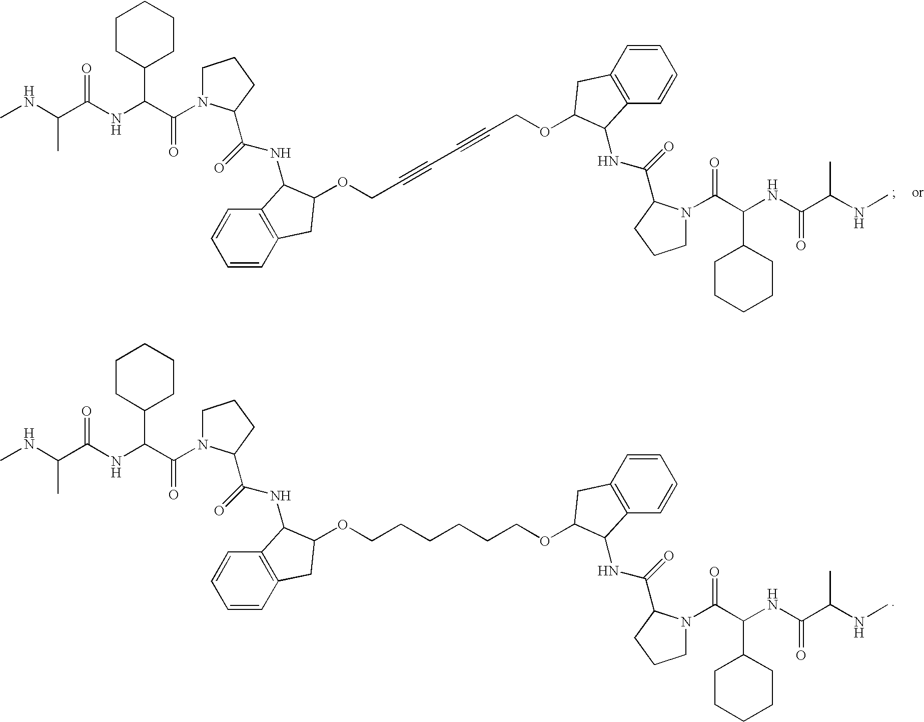

In embodiments of any of the compounds, compounds of formula I can have the stereochemistry shown below in formula Ia:

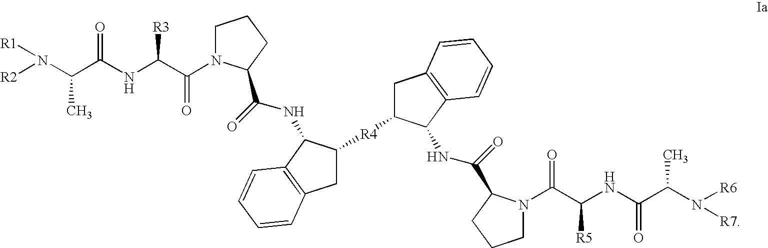

In other embodiments of any of the compounds, compounds of formula I can have the stereochemistry shown below in formula Ib:

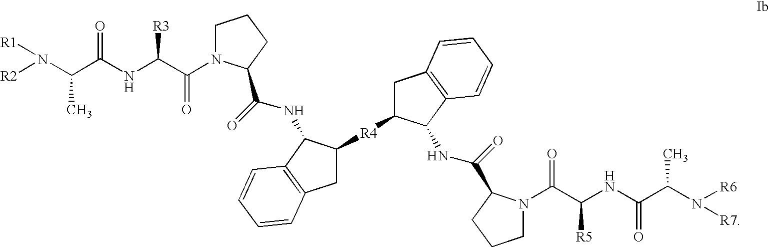

Specific examples of the compounds described herein are set forth in Table 1. Those skilled in the art recognize that the compounds described herein, including those set forth in Table 1 and the Examples, can occur in the free, non-salt, form or can occur as salts.

Those skilled in the art will recognize that the compounds described herein can be considered dimeric. The dimeric compounds described herein can be homodimers or heterodimers. The terms homodimer and heterodimer describe dimers that contain two identical subunits or two different subunits, respectively. The two subunits are linked by a linker moiety, i.e. R4, wherein the linker moiety is covalently bonded to each of the subunits at the indicated position. Accordingly, in homodimers of the described compounds, R1, R2 and R3 are the same as R6, R7 and R5, respectively, with the linker being R4. In heterodimers of the described compounds, one or more R1, R2 and R3 are different than R6, R7 and R5, respectively. In heterodimers, one, two or all of R1, R2 and R3 can be different than R6, R7 and R5, respectively.

Included within the scope of the compounds of Formula I are all isomers (e.g. enantiomers, stereoisomers, diastereoisomers, epimers, geometrical isomers) of the compounds described herein alone as well as any mixtures, such as wholly or partially racemized or epimerized (e.g. racemic or optically active mixtures), of the compounds of Formula I. All of these forms, including (R), (S), epimers, diastereomers, cis, trans, syn, anti, and mixtures thereof, are included in the compounds of Formula I. Compounds described herein may exist in hydrated, solvated, tautomeric, or Zwitterionic form and the compounds include any of these forms of the compounds, and mixtures thereof. The compounds of Formula I can be provided as salts, for example pharmaceutically acceptable salts, and can also take the form of clathrates.

Stereoisomeric mixtures, e.g. mixtures of diastereomers, can be separated into their corresponding isomers in a known manner by means of suitable separation methods. Diastereomeric mixtures for example can be separated into their individual diastereomers by means of fractional crystallization, chromatography, solvent distribution, and similar procedures. This separation can take place either at the level of one of the starting compounds, intermediate compounds or of a compound itself. Enantiomers can be separated through the formation of diastereomeric salts, for example by salt formation with an enantiomerically pure chiral acid, or by means of chromatography, for example by HPLC, using chiral chromatographic media.

In a specific embodiment, the compounds of formula I can be a mixture of any form of the compound, for example as shown in formula Ia or as set forth in an Example, in the presence or absence of the other forms of the compound of formula I. Mixtures of the compounds shown include racemic or equimolar mixtures as well as mixtures of the compounds where one of the forms is enriched relative to the other isomers, for example a 3:2 mixture in which the compound shown in formula Ia or as set forth in an Example, is the major isomer. In additional embodiments, any form of the compound of formula I, such as the form of the compound shown in formula Ia or as set forth in an Example, can make up about 60, 70, 80, 85, 90, 95, 97, 99, 99.5, 99.7, 99.9 percent or more of the mixture of the compounds of formula I on a molar or weight basis. In one embodiment, a specific form of the compound can make up about 90 percent or more of the mixture of the compounds of formula I. In an additional embodiment, a specific form of the compound can make up about 95 percent or more of the mixture of the compounds of formula I. In still a further embodiment, a specific form of the compound can make up about 99 percent or more of the mixture of the compounds of formula I.

It is understood that the compounds described herein may exhibit the phenomenon of tautomerism. As the chemical structures sometimes only represent one of the possible tautomeric forms, it should be understood that the invention encompasses any tautomeric form of the represented structure.

In addition, the compounds described herein can exist in unsolvated as well as solvated forms with solvents, including pharmaceutically acceptable solvents such as water, ethanol, and the like. In general, the solvated forms are considered equivalent to the unsolvated forms for the purposes of the present invention and are included within the compounds of Formula I. The compounds can also be present in complexes such as clathrates, drug-host inclusion complexes wherein the drug and host can be present in stoichiometric or non-stoichiometric amounts. Included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

Any embodiment described herein can be combined with any other suitable embodiment described herein to provide additional embodiments. For example, where one embodiment individually or collectively describes possible groups for R1, R2, R3, R4, R5, R6, etc., and a separate embodiment describes possible R7 groups, it is understood that these embodiments can be combined to provide an embodiment describing possible groups for R1, R2, R3, R4, R5, R6, with the possible R7 groups, etc.

With respect to the above compounds, and throughout the application and claims, the following terms have the meanings defined below.

The term “alkenyl” refers to straight and branched chain hydrocarbons, such as those described with respect to alkyl groups described herein, that include at least one double bond existing between two carbon atoms. Alkenyl groups can be monovalent or divalent, as appropriate. Examples include vinyl, —CH═C(H)(CH₃), —CH═C(CH₃)₂, —C(CH₃)═C(H)₂, —C(CH₃)═C(H)(CH₃), —C(CH₂CH₃)═CH₂, —C(CH₂CH₃)═CH—, butadienyl, pentadienyl, and hexadienyl among others.

The term “alkyl” refers to saturated hydrocarbon chains, for example C(1-6) chains, that do not contain heteroatoms. Alkyl groups can be monovalent or divalent, as appropriate. Thus, the phrase includes straight chain alkyl groups such as methyl, ethyl, propyl, butyl, pentyl, hexyl, heptyl, octyl, nonyl, decyl, undecyl, dodecyl, —CH₂CH₂—, and the like. The phrase also includes branched chains, including, but not limited to, the following which are provided by way of example: —(CH₂)₆—, —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃), —CH₂CH₂C(CH₂CH₃)₃, —CH(CH₃)CH₂CH(CH₃)₂, —CH(CH₃)CH(CH₃)CH(CH₃)₂, —CH(CH₂CH₃)CH(CH₃)CH(CH₃)(CH₂CH₃), and others. The phrase includes primary alkyl groups, secondary alkyl groups, and tertiary alkyl groups. Alkyl groups can be bonded to one or more carbon atom(s), oxygen atom(s), nitrogen atom(s), and/or sulfur atom(s) in the parent compound.

The term “alkynyl” refers to straight and branched chain hydrocarbon groups, such as those described with respect to alkyl groups as described herein, except that at least one triple bond exists between two carbon atoms. Alkynyl groups can be monovalent or divalent, as appropriate. Examples include —C≡C(H), —C≡C(CH₃), —C≡C—, —C≡CCH₂—, —C≡C(CH₂CH₃), —C(H₂)C≡C(H), —C(H)₂C≡C(CH₃), —C(H)₂C≡C(CH₂CH₃) and —CH₂—C≡C—C≡C—CH₂—, among others.

The term “cycloalkyl” refers to saturated cyclic hydrocarbon chains, generally having from 3 to 12 carbon atoms, and includes cyclic alkyl groups such as cyclopropyl, cyclobutyl, cyclopentyl, cyclohexyl, cycloheptyl, and cyclooctyl. The phrase also includes polycyclic alkyl groups such as, but not limited to, adamantyl, norbornyl, and bicyclo[2.2.2]octyl. Cycloalkyl groups can be bonded to one or more carbon atom(s), oxygen atom(s), nitrogen atom(s), and/or sulfur atom(s) in the parent compound.

As used herein, “IAP” or “IAPs” designates one or more of the art-recognized inhibitor of apoptosis proteins, which include cIAP1, cIAP2, XIAP, Ts-IAP, Livin, survivin, NAIP and Apollon or Bruce.

“Pharmaceutically acceptable” means suitable for use in mammals. Pharmaceutically acceptable salts include salts with an inorganic base, organic base, inorganic acid, organic acid, or basic or acidic amino acid suitable for use in mammals. Salts of inorganic bases include alkali metal ions such as sodium or potassium; alkaline earth metals such as calcium and magnesium or aluminum; and ammonium. Organic bases include trimethylamine, triethylamine, pyridine, picoline, ethanolamine, diethanolamine, and triethanolamine. Inorganic acids include hydrochloric acid, hydroboric acid, nitric acid, sulfuric acid, and phosphoric acid. Organic acids include for example, formic acid, acetic acid, trifluoroacetic acid, fumaric acid, oxalic acid, tartaric acid, maleic acid, citric acid, succinic acid, malic acid, methanesulfonic acid, ethanesulfonic acid, benzenesulfonic acid, and p-toluenesulfonic acid. Basic amino acids include arginine, lysine and ornithine. Acidic amino acids include, for example, aspartic acid and glutamic acid. Examples of pharmaceutically acceptable salts are also described in Berge, S. M. et al., “Pharmaceutical Salts,” Journal of Pharmaceutical Science, 1977; 66:1 19.

A “salt” refers to all salt forms of a compound, including salts suitable for use in industrial processes, such as the preparation of the compound, and pharmaceutically acceptable salts.

“Treat” or “Treating” means treatment of a disease-state associated with insufficient apoptosis related to an IAP in a subject. Accordingly, treat or treating includes inhibiting a disease or condition associated with insufficient apoptosis related to an IAP, i.e., arresting its development and/or relieving a disease or condition associated with insufficient apoptosis related to an IAP, i.e., causing regression or alleviation of the condition or any symptom thereof. When the disease-state associated with insufficient apoptosis related to an IAP is cancer, treat or treating includes an alleviation of the cancer, e.g. any symptom of the cancer, by killing, inhibiting the growth, and/or inhibiting the metastasis of the cancer cells.

Compounds described herein can be provided ex vivo or, in some instances, produced in vivo, for example where a prodrug of a compound is administered.

Generally, reference to a certain element such as hydrogen or H is meant to include all isotopes of that element. For example, if an R group is defined to include hydrogen or H, it also includes deuterium and tritium.

Certain compounds described herein are also useful as intermediates for preparing other described compounds and such intermediates are included within the scope of the present invention.

Specific compounds are described throughout with particular reference to the Examples and in the following table:

TABLE 1

| | |
|---|---|
| Cpd./Ex. no. 1 | Name: (S,S,2S,2′S)-N,N′-((1S,1′S,2R,2′R)-2,2′-(hexa-2,4-diyne-1,6- |
| | diylbis(oxy))bis(2,3-dihydro-1H-indene-2,1-diyl))bis(1-((S)-2-cyclohexyl-2-((S)- |
| | 2-(methylamino)propanamido)acetyl)pyrrolidine-2-carboxamide) |

Structure:

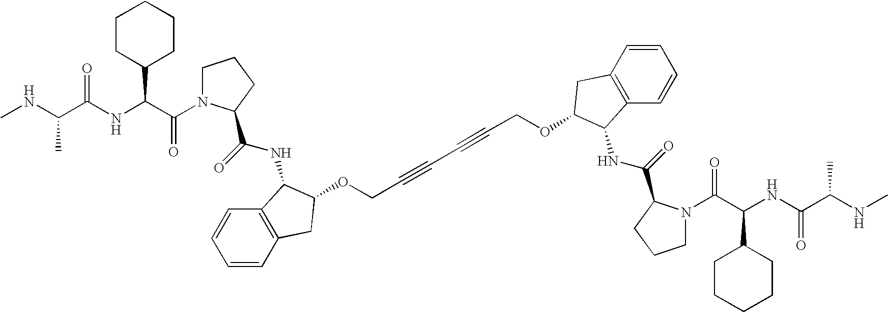

| | |
|---|---|
| Cpd./Ex. no. 1c | Name: (2S,2′S)-N,N′-{hexa-2,4-diyne-1,6-diylbis[oxy(1S,2R)-2,3-dihydro-1H- |
| | indene-2,1-diyl]}bis{1-[(2S)-2-cyclohexyl-2-({(2S)-2- |
| | [(¹³C,²H₃)methylamino]propanoyl}amino)acetyl]pyrrolidine-2-carboxamide} |
| | as a dibenzoate salt |

Structure:

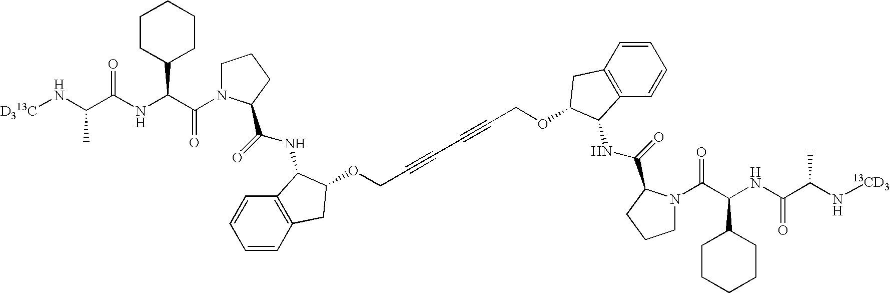

| | |
|---|---|
| Cpd./Ex. no. 2 | Name: (S)-1-((S)-2-cyclohexyl-2-((S)-2-(methylamino)propanamido)acetyl)-N- |
| | ((1S,2R)-2-(prop-2-ynyloxy)-2,3-dihydro-1H-inden-1-yl)pyrrolidine-2-carboxamide |

Structure:

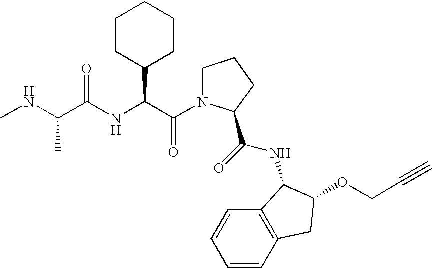

| | |
|---|---|
| Cpd./Ex. no. 3 | Name: (S,S,2S,2′S)-N,N′-((1S,1′S,2R,2′R)-2,2′-(Hexane-1,6- |
| | diylbis(oxy))bis(2,3-dihydro-1H-indene-2,1-diyl))bis(1-((S)-2-cyclohexyl-2-((S)- |
| | 2-(methylamino)propanamido)acetyl)pyrrolidine-2-carboxamide) as a bis hydrochloride salt |

Structure:

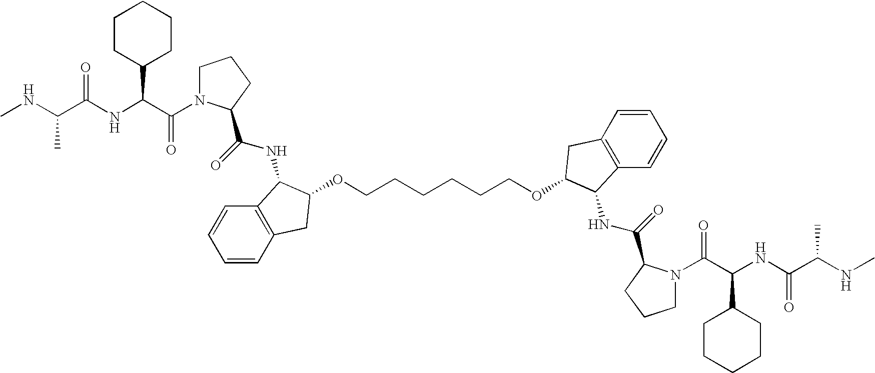

| | |
|---|---|
| Cpd./Ex. no. 4 | Name: (S,S,2S,2′S)-N,N′-((1S,1′S,2S,2′S)-2,2′-(hexa-2,4-diyne-1,6- |
| | diylbis(oxy))bis(2,3-dihydro-1H-indene-2,1-diyl))bis(1-((S)-2-cyclohexyl-2-((S)- |
| | 2-(methylamino)propanamido)acetyl)pyrrolidine-2-carboxamide) |

Structure:

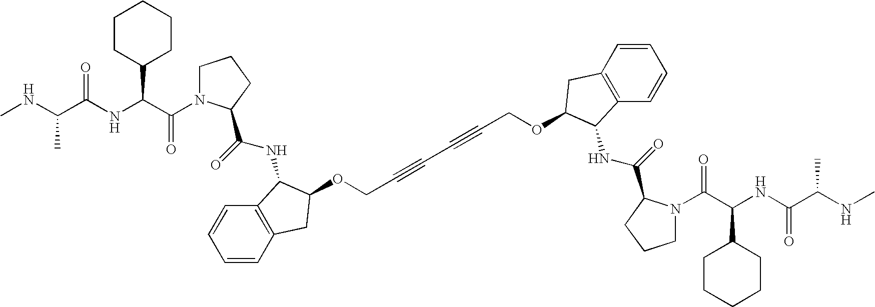

Also provided are pharmaceutical compositions comprising one or more of the compounds herein, or pharmaceutically acceptable salts or tautomers thereof, with one or more pharmaceutically acceptable carriers, excipients, binders, diluents or the like. The pharmaceutically acceptable compositions, which can contain a therapeutically effective dose of one or more of the compounds, can be used to treat or ameliorate a variety of conditions or diseases. A therapeutically effective dose or amount refers to that amount of one or more compounds described herein sufficient to treat cancer.

The pharmaceutical compositions of the instant invention can be manufactured by methods well known in the art such as conventional granulating, mixing, dissolving, encapsulating, lyophilizing, emulsifying or levigating processes, among others. The compositions can be in the form of, for example, granules, powders, tablets, capsule syrup, suppositories, injections, emulsions, elixirs, suspensions or solutions. The instant compositions can be formulated for various routes of administration, for example, by oral administration, topical administration, by transmucosal administration, by rectal administration, by intravaginal administration or subcutaneous administration as well as intrathecal, intravenous, intramuscular, intraperitoneal, intranasal, intraocular or intraventricular injection. The compound or compounds of the instant invention can also be administered in a local rather than a systemic fashion, such as injection as a sustained release formulation or topically. The following dosage forms are given by way of example and should not be construed as limiting the instant invention.

For oral, buccal, and sublingual administration, powders, suspensions, granules, tablets, pills, capsules, gelcaps, and caplets are acceptable as solid dosage forms. These can be prepared, for example, by mixing one or more compounds of the instant invention, or pharmaceutically acceptable salts or tautomers thereof, with at least one additive or excipient such as a starch or other additive. Suitable additives or excipients are sucrose, lactose, cellulose sugar, mannitol, maltitol, dextran, sorbitol, starch, agar, alginates, chitins, chitosans, pectins, tragacanth gum, gum arabic, gelatins, collagens, casein, albumin, synthetic or semi-synthetic polymers or glycerides, methyl cellulose, hydroxypropylmethyl-cellulose, and/or polyvinylpyrrolidone. Optionally, oral dosage forms can contain other ingredients to aid in administration, such as an inactive diluent, or lubricants such as magnesium stearate, or preservatives such as paraben or sorbic acid, or antioxidants such as ascorbic acid, tocopherol or cysteine, a disintegrating agent, binders, thickeners, buffers, sweeteners, flavoring agents or perfuming agents. Additionally, dyestuffs or pigments can be added for identification. Tablets and pills can be further treated with suitable coating materials known in the art.

Liquid dosage forms for oral administration can be in the form of pharmaceutically acceptable emulsions, syrups, elixirs, suspensions, slurries and solutions, which can contain an inactive diluent, such as water. Pharmaceutical formulations can be prepared as liquid suspensions or solutions using a sterile liquid, such as, but not limited to, an oil, water, an alcohol, and combinations of these. Pharmaceutically suitable surfactants, suspending agents, emulsifying agents, can be added for oral or parenteral administration.

As noted above, suspensions can include oils. Such oils include peanut oil, sesame oil, cottonseed oil, corn oil, olive oil and mixtures of oils. Suspension preparation can also contain esters of fatty acids such as ethyl oleate, isopropyl myristate, fatty acid glycerides and acetylated fatty acid glycerides. Suspension formulations can include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water can also be used in suspension formulations.

The provided compounds can also be administered topically to the skin or mucosa, that is, dermally or transdermally. Typical topical formulations include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated.

For inhaled or nasal administration, the pharmaceutical formulations can be a spray or aerosol containing and appropriate solvents and optionally other compounds such as, but not limited to, stabilizers, antimicrobial agents, antioxidants, pH modifiers, surfactants, bioavailablity modifiers and combinations of these. A propellant for an aerosol formulation can include compressed air, nitrogen, carbon dioxide, or a hydrocarbon based low boiling solvent. The compound or compounds of the instant invention are conveniently delivered in the form of an aerosol spray presentation from a nebulizer or the like.

Injectable dosage forms generally include aqueous suspensions or oil suspensions which can be prepared using a suitable dispersant or wetting agent and a suspending agent. Injectable forms can be in solution phase or in the form of a suspension, prepared with a solvent or diluent. Acceptable solvents or vehicles include sterilized water, Ringer's solution, or an isotonic aqueous saline solution. Alternatively, sterile oils can be employed as solvents or suspending agents. Generally, the oil or fatty acid is non-volatile, including natural or synthetic oils, fatty acids, mono-, di- or tri-glycerides.

For injection, the pharmaceutical formulation can be a powder suitable for reconstitution with an appropriate solution as described above. Examples of these include freeze dried, rotary dried or spray dried powders, amorphous powders, granules, precipitates, or particulates. For injection, the formulations can optionally contain stabilizers, cyclodextrins, such as a beta-cyclodextrin, pH modifiers, surfactants, bioavailability modifiers and combinations of these. The compounds can be formulated for parenteral administration by injection such as by bolus injection or continuous infusion. A unit dosage form for injection can be in ampoules or in multi-dose containers.

For rectal or intravaginal administration, the pharmaceutical formulations can be in the form of a suppository, pessary, ointment, enema, a tablet or a cream for release of compound, such as in the intestines, sigmoid flexure and/or rectum. Rectal suppositories are prepared by mixing one or more compounds of the instant invention, or pharmaceutically acceptable salts or tautomers of the compound, with acceptable vehicles, for example, cocoa butter or polyethylene glycol, which is present in a solid phase at normal storing temperatures, and present in a liquid phase at those temperatures suitable to release a drug inside the body, such as in the rectum. Oils can also be employed in the preparation of formulations of the soft gelatin type and suppositories. Water, saline, aqueous dextrose and related sugar solutions, and glycerols can be employed in the preparation of suspension formulations which can also contain suspending agents such as pectins, carbomers, methyl cellulose, hydroxypropyl cellulose or carboxymethyl cellulose, as well as buffers and preservatives.

Besides those representative dosage forms described above, pharmaceutically acceptable excipients and carriers are generally known to those skilled in the art and are thus included in the instant invention. Such excipients and carriers are described, for example, in “Remingtons Pharmaceutical Sciences” Mack Pub. Co., New Jersey (1991).

The formulations of the invention can be designed for to be short-acting, fast-releasing, long-acting, and sustained-releasing. Thus, the pharmaceutical formulations can also be formulated for controlled release or for slow release.

The instant compositions can also comprise, for example, micelles or liposomes, or some other encapsulated form, or can be administered in an extended release form to provide a prolonged storage and/or delivery effect. Therefore, the pharmaceutical formulations can be compressed into pellets or cylinders and implanted intramuscularly or subcutaneously as depot injections or as implants such as stents. Such implants can employ known materials such as silicones and biodegradable polymers.

The compositions can contain, for example, from about 0.1 percent by weight, to about 99 percent or more by weight, of the active material, depending on the method of administration. Where the compositions comprise dosage units, each unit can contain, for example, from about 1 to about 3000 mg or more of the active ingredient. The dosage as employed for adult human treatment can range, for example, from about 1 to about 3000 mg per day, depending on the route and frequency of administration.

Specific dosages can be adjusted depending on conditions of infection, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant invention.

A therapeutically effective dose or amount can vary depending upon the route of administration and dosage form. Some compositions of the instant invention can provide a formulation that exhibits a high therapeutic index. The therapeutic index is the dose ratio between toxic and therapeutic effects which can be expressed as the ratio between LD₅₀ and ED₅₀. The LD₅₀ is the dose lethal to 50 percent of the population and the ED₅₀ is the dose therapeutically effective in 50 percent of the population. The LD₅₀ and ED₅₀ can be determined by standard pharmaceutical procedures in animal cell cultures or experimental models.

The compounds of the invention are believed to inhibit the binding of IAP proteins to caspases. The compounds of the invention are also believed to cause the release of caspases from inhibitory IAP protein:caspase complexes, thereby promoting caspase enzymatic activity. In addition, the compounds of the invention are believed to bind to and directly promote the degradation of the IAP proteins cIAP-1 and cIAP-2. The degradation of cIAP-1 and cIAP-2 proteins is believed to promote apoptosis in response to activation of the TNF receptor superfamily, which includes receptors for the ligands Trail and TNFalpha. Accordingly, the compounds of the invention are useful for inducing apoptosis in cells or sensitizing cells to apoptotic signals, in particular cancer cells. Compounds of the invention are useful for inducing apoptosis in cells that overexpress IAP proteins. More broadly, the compounds can be used for the treatment of all cancer types which fail to undergo apoptosis. Examples of such cancer types include neuroblastoma, intestine carcinoma such as rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, craniopharyngeoma, osteosarcoma, chondrosarcoma, myosarcoma, liposarcoma, fibrosarcoma, Ewing sarcoma and plasmocytoma.

In particular, the compounds, compositions and methods of the present invention can be used for the treatment of cancer, including solid tumors, such as bladder, breast, colon, or ovarian cancer. The compounds, compositions and methods of the present invention can also be used for the treatment of AML, diffuse large B-cell lymphoma (DLBCL), non-small cell lung cancer (NSCLC), including the non-squamous and squamous subtypes, pancreatic, or prostate cancer.

Another embodiment provides a method of inhibiting the binding of IAP proteins to caspases with either a non-therapeutic amount or a therapeutically effective amount of one or more of the present compounds. Such methods can occur in vivo or in vitro. In vitro contact can involve a screening assay to determine the efficacy of the one or more compounds against selected target, tissue, or tumor at various amounts or concentrations. In vivo contact with a therapeutically effective amount of the one or more compounds can involve testing or treatment of cancer in the animal in which the contact occurs. The effect of the one or more compounds on the target or host animal can also be determined or measured.

Accordingly, one embodiment provides a compound described herein, or a pharmaceutically acceptable salt thereof, for use as a medicament.

In one embodiment, the invention provides methods of treating cancer in a subject, such as a mammal, e.g., a human or non-human mammal, comprising administering an effective amount of one or more compounds described herein to the subject. Suitable subjects that can be treated include domestic or wild animals, companion animals, such as dogs, cats and the like; livestock, including horses, cows and other ruminants, pigs, poultry, rabbits and the like; primates, for example monkeys, such as rhesus monkeys and cynomolgus (also known as crab-eating or long-tailed) monkeys, marmosets, tamarins, chimpanzees, macaques and the like; and rodents, such as rats, mice, gerbils, guinea pigs and the like. In one embodiment, the compound is administered in a pharmaceutically acceptable form, optionally in a pharmaceutically acceptable carrier.

Also provided is an article of manufacture comprising a pharmaceutical composition comprising a provided compound contained within a packaging material and a label or package insert which indicates that said pharmaceutical composition can be used for treating cancer, as described herein.

The compounds described herein may be used in the methods described herein as either a single agent by itself or in combination with other agents. One or more of these compounds could also prevent the potential cancer resistance mechanisms that may arise due to mutations in a set of genes. For example, the cancer treatment defined herein may be applied as a sole therapy or may involve, in addition to the compound described herein, conventional surgery or radiotherapy or chemotherapy. Such chemotherapy may include one or more of the following categories of anti-tumor agents:

(i) other antiproliferative/antineoplastic drugs and combinations thereof, as used in medical oncology, such as alkylating agents (for example cis platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5 fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine; and taxoids like taxol and taxotere; and polo kinase or kinesin motor protein inhibitors); and topoisomerase inhibitors (for example epipodophyllotoxins like etoposide and teniposide, amsacrine, topotecan, camptothecin and irinotecan);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5*-reductase such as finasteride;

(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase, inhibitors of FAK or focal-adhesion kinase, inhibitors of or antibodies to MET receptor kinase or the MET ligand hepatocyte growth factor;

(iv) inhibitors of growth factor function: for example such inhibitors include growth factor antibodies and growth factor receptor antibodies (for example the anti erbB2 antibody trastuzumab [Herceptin™], the anti-EGFR antibody panitumumab, the anti erbB1 antibody cetuximab [Erbitux, C225] and any growth factor or growth factor receptor antibodies disclosed by Stern et al. (Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI 774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R75777) and lonafarnib (SCH66336)), inhibitors of cell signaling through MEK and/or AKT kinases, c-kit inhibitors, abl kinase inhibitors, PI3 kinase inhibitors, Flt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;

(v) antiangiogenic agents such as those which inhibit the effects of vascular endothelial growth factor, [for example the anti vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin alphavbeta3 function and angiostatin)];

(vi) vascular damaging agents such as Combretastatin A4 and compounds disclosed in International Patent Applications WO 99/02166, WO 00/40529, WO 00/41669, WO 01/92224, WO 02/04434 and WO 02/08213;

(vii) an endothelin receptor antagonist, for example zibotentan (ZD4054) or atrasentan;

(viii) antisense therapies, for example those which are directed to the targets listed above, such as ISIS 2503, an anti-ras antisense; or oblimerson sodium, an anti-Bc1-2 antisense;

(ix) gene therapy approaches, including for example approaches to replace aberrant genes such as aberrant p53 or aberrant BRCA1 or BRCA2, GDEPT (gene directed enzyme pro drug therapy) approaches such as those using cytosine deaminase, thymidine kinase or a bacterial nitroreductase enzyme and approaches to increase patient tolerance to chemotherapy or radiotherapy such as multi drug resistance gene therapy;

(x) immunotherapy approaches, including for example ex vivo and in vivo approaches to increase the immunogenicity of patient tumor cells, such as transfection with cytokines such as interleukin 2, interleukin 4 or granulocyte macrophage colony stimulating factor, approaches to decrease T cell anergy, approaches using transfected immune cells such as cytokine transfected dendritic cells, approaches using cytokine transfected tumor cell lines, approaches using anti idiotypic antibodies, approaches for T-cell enhancement including CTLA4 antibodies, and antibodies directed toward CD137, PD-1 or B7-H1, toll-receptor agonists;

(xi) pro-apoptotic approaches, including antibodies to death receptor 4 or death receptor 5 or antibodies binding to both death receptor 4 and death receptor 5;

(xii) cytokine treatment, including tumor necrosis factor alpha, and recombinant Trail protein or small molecule or protein mimetics of the Trail protein;

(xiii) efficacy enhancers, such as leucovorin; and

(xiv) radiation.

Such conjoint treatment may be achieved by way of the simultaneous, sequential or separate dosing of the individual components of the treatment. Such combination products employ the compounds described herein, or pharmaceutically acceptable salts thereof, within the dosage range described herein and the other pharmaceutically active agent, typically within its approved dosage range.

According to this aspect there is provided a combination suitable for use in the treatment of cancer comprising a compound described herein or a pharmaceutically acceptable salt thereof, and any one or more of the agents listed under (i)-(xiv) above. A described combination can be used for the manufacture of a medicament for use in the treatment of cancer in a mammal for example in a human.

As is understood by those skilled in the art, the term combination is used it is to be understood that this refers to simultaneous, separate or sequential administration. Where the administration is sequential or separate, the delay in administering the second component should not be such as to lose the beneficial effect of the combination.

According to a further aspect a compound described herein or a pharmaceutically acceptable salt thereof can be combined in a pharmaceutical composition with one or more agents described in (i)-(xiv) above with a pharmaceutically acceptable diluent or carrier. Such pharmaceutical compositions can be used in inducing apoptosis, e.g., in the treatment of cancer.

### EXAMPLES

The following descriptions of experiments, procedures, examples, and intermediates are intended to exemplify embodiments of the invention, and are in no way intended to be limiting.

The invention will now be illustrated by the following non-limiting examples in which, unless stated otherwise:

(i) temperatures are given in degrees Celsius (° C.); operations were carried out at room or ambient temperature, that is, at a temperature in the range of 18 to 25° C. unless otherwise indicated;

(ii) organic solutions were dried over anhydrous sodium sulphate; evaporation of solvent was carried out using a rotary evaporator under reduced pressure with a heating bath, or a centrifugal evaporator, e.g. Genevac;

(iii) in general, the course of reactions was followed by TLC or LCMS and reaction times are given for illustration only;

(iv) final products had satisfactory proton nuclear magnetic resonance (NMR) spectra and/or mass spectral data;

(v) yields are given for illustration only and are not necessarily those which can be obtained by diligent process development; preparations were repeated if more material was required;

(vi) when given, NMR data is in the form of delta values for major diagnostic protons, given in parts per million (ppm) relative to tetramethylsilane (TMS) as an internal standard, determined at 400 or 300 MHz using perdeuterio dimethyl sulphoxide (DMSO-d6) as solvent unless otherwise indicated and spectra were recorded at temperatures between ambient temperature and 100° C.;

(vii) chemical symbols have their usual meanings; SI units and symbols are used;

(viii) solvent ratios are given in volume:volume (v/v) terms; and

(ix) mass spectra were acquired when samples were separated using reverse-phase liquid chromatography (LC) and detected by electrospray ionization (ESI) mass spectrometry (MS) in positive and negative ion; values for m/z are given; generally, only ions which indicate the parent mass are reported; and unless otherwise stated, the mass ion quoted is (MH)+;

(x) where a synthesis is described as being analogous to that described in a previous example the amounts used are the millimolar ratio equivalents to those used in the previous example;

(xi) “Isco” refers to normal phase flash column chromatography using pre-packed silica gel cartridges used according to the manufacturers instruction; and

(xii) “Gilson” refers to reverse phase HPLC. Mobile phases include water, CH₃CN, MeOH and THF. Modifiers include 0.1% trifluoroacetic acid (TFA), 0.1% formic acid, 0.2% NH₄OH or 10 mM ammonium acetate. Columns include XBridge C18 OBD, 19×100 mm, 5 μm, Atlantis T3, 19×100 mm, 5 μm.

### INTERMEDIATES

### Intermediate 1

### (1S,2R)-2-(Prop-2-ynyloxy)-2,3-dihydro-1H-inden-1-amine

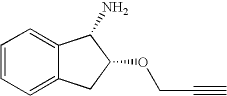

Sodium hydride (60% dispersion in mineral oil; 977 mg, 24.4 mmol) was washed by mixing with hexanes, allowing to settle and removing the supernatant via syringe. Tetrahydrofuran (THF) (60 mL) was then added, and the suspension was cooled to 0° C. (1S,2R)-(−)-cis-1-Amino-2-indanol (Aldrich; 3.00 g, 20.11 mmol) was added in portions over 5 minutes (min) (gas evolution observed). The resulting mixture was warmed to room temperature until hydrogen evolution ceased, and the resulting thick suspension was heated to 70° C. A solution of propargyl bromide (80% in toluene; 2.50 mL, 22.4 mmol) in THF (20 mL) was added dropwise over 20 min. The reaction mixture was heated to reflux for 45 min. The reaction was quenched with H₂O and extracted with ethyl acetate (EtOAc). The organics were washed with saturated (sat) NaCl and then dried with solid (s) Na₂SO₄. The solvents were removed under reduced pressure. The crude material was carried on without any further purification; m/z 188.

### Intermediate 2

### (S)-tert-Butyl 2-((1S,2R)-2-(prop-2-ynyloxy)-2,3-dihydro-1H-inden-1-ylcarbamoyl)pyrrolidine-1-carboxylate

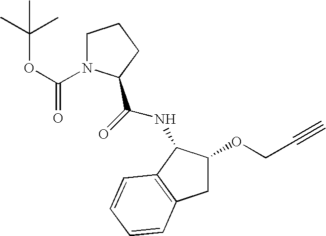

A solution of BOC-L-proline (4.46 g, 20.72 mmol) and 4-methylmorpholine (2.50 mL, 22.7 mmol) in EtOAc (50 mL) was treated with ethyl chloroformate (2.00 mL, 20.9 mmol) at 0° C. The reaction mixture was stirred for 20 min, and then a solution of (1S,2R)-2-(prop-2-ynyloxy)-2,3-dihydro-1H-inden-1-amine (Intermediate 1, 3.76 g, 20.1 mmol) in EtOAc (30 mL) was added. The resulting mixture was warmed to room temperature and stirred overnight. The reaction was quenched with water and extracted with EtOAc. The organics were washed with 1 N HCl and then NaHCO₃(sat). The solvents were removed under reduced pressure. The crude material was purified by an Isco system using a gradient elution of hexanes/EtOAc (silica cartridge) to give the desired product. NMR (dimethylsulfoxide-d6 [DMSO-d6]) 8.05 (d, J=8.34 Hz, 1H), 7.19-7.28 (m, 4H), 5.29-5.33 (m, 1H), 4.31-4.36 (m, 1H), 4.20-4.29 (m, 1H), 4.15-4.18 (m, 2H), 3.34-3.45 (m, 2H), 3.21-3.34 (m, 2H), 3.00-3.06 (m, 2H), 2.02-2.16 (m, 1H), 1.73-1.98 (m, 4H), 1.32 (s, 9H); m/z 385.

### Intermediate 3, alternate 1

### (S)—N-((1S,2R)-2-(Prop-2-ynyloxy)-2,3-dihydro-1H-inden-1-yl)pyrrolidine-2-carboxamide hydrochloride

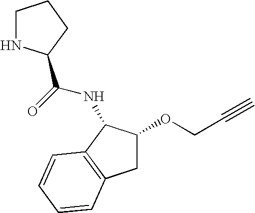

(S)-tert-Butyl 2-((1S,2R)-2-(prop-2-ynyloxy)-2,3-dihydro-1H-inden-1-ylcarbamoyl)pyrrolidine-1-carboxylate (Intermediate 2, 3.86 g, 10.04 mmol) was treated with 4 N HCl in dioxane (15.0 mL). The resulting solution was stirred at room temperature for 1 hour (h). The solvents were removed under reduced pressure. The resulting product was dried in vacuo overnight to give the title compound. NMR (DMSO-6) 10.06 (bs, 1H), 8.75-8.77 (m, 1H), 8.51-8.60 (m, 1H), 7.21-7.29 (m, 4H), 5.35-5.39 (m, 1H), 4.35-4.39 (m, 1H), 4.23-4.29 (m, 1H), 4.17-4.19 (m, 2H), 3.42-3.48 (m, 1H), 3.36-3.42 (m, 1H), 3.25-3.33 (m, 1H), 3.14-3.24 (m, 2H), 2.97-3.11 (m, 2H), 2.20-2.35 (m, 1H), 1.82-2.02 (m, 4H); m/z 285.

### Intermediate 3, alternate 2

### (2S)-N-[(1S,2R)-2-prop-2-ynoxyindan-1-yl]pyrrolidine-2-carboxamide

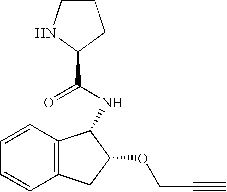

Intermediate 13 (1341 g, 3.49 moles) was dissolved in dichloromethane (DCM) (4.5 L). Trifluoroacetic acid (2.25 L, 30.29 moles) was added over 10 minutes and the resulting solution stirred at room temperature for 2 hours. The solvent was removed by evaporation under reduced pressure. Diethyl ether (11 L) was added to the residue and the mixture stirred for 1 hour at below 10° C. The resulting solid was collected by filtration, washed with diethyl ether (1 L) then dried at 30° C. under reduced pressure to give intermediate 13 as a solid in 93% yield (1293 g) with a purity of 97.9% by HPLC. 1H NMR (400 MHz, DMSO-d6) δ ppm 9.01 (br. s., 1H) 8.74 (d, 1H) 7.21-7.29 (m, 4H) 5.38 (dd, 1H) 4.36-4.40 (m, 1H) 4.26-4.29 (m, 1H) 4.18 (d, 2H) 3.45 (t, 1H) 3.27-3.36 (m, 1H) 3.17-3.27 (m, 1H) 2.95-3.13 (m, 2H) 2.23-2.36 (m, 1H) 1.94-2.03 (m, 1H) 1.85-1.94 (m, 2H). m/z 285.

### Intermediate 4

### tert-Butyl (S)-1-((S)-1-cyclohexyl-2-oxo-2-((S)-2-((1S,2R)-2-(prop-2-ynyloxy)-2,3-dihydro-1H-inden-1-ylcarbamoyl)pyrrolidin-1-yl)ethylamino)-1-oxopropan-2-yl(methyl)carbamate

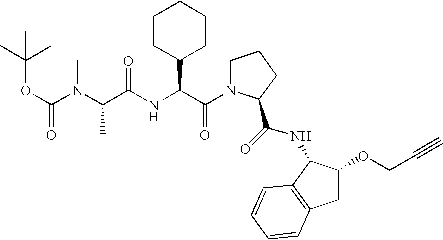

A solution of (S)-2-((S)-2-(tert-butoxycarbonyl(methyl)amino)propanamido)-2-cyclohexylacetic acid (Intermediate 7, 430 mg, 1.26 mmol), (S)—N-((1S,2R)-2-(prop-2-ynyloxy)-2,3-dihydro-1H-inden-1-yl)pyrrolidine-2-carboxamide hydrochloride (Intermediate 3, 391 mg, 1.22 mmol) and 4-methylmorpholine (150 μL, 1.36 mmol) in EtOAc (8.0 mL) was treated with 4-(4,6-dimethoxy-1,3,5-triazine-2-yl)-4-methyl morpholinium chloride (DMTMM) (370 mg, 1.34 mmol) at 0° C. The solution was warmed to room temperature and stirred overnight. The reaction mixture was quenched with H₂O and extracted with EtOAc. The solvents were removed under reduced pressure and the residue was purified by an Isco system using a hexanes/EtOAc gradient (silica cartridge) to give the desired product (469 mg, 63%). NMR (DMSO-d6, 100° C.) 7.58-7.63 (m, 1H), 7.07-7.25 (m, 5H), 5.32-5.35 (m, 1H), 4.51-4.58 (m, 1H), 4.45-4.47 (m, 1H), 4.34-4.42 (m, 1H), 4.14-4.24 (m, 2H), 3.72-3.78 (m, 1H), 3.54-3.63 (m, 1H), 3.16-3.19 (m, 1H), 3.04-3.05 (m, 2H), 2.76 (s, 3H), 1.97-2.09 (m, 3H), 1.83-1.93 (m, 1H), 1.58-1.78 (m, 7H), 1.44 (s, 9H), 1.25 (d, 4H), 0.91-1.21 (m, 5H); m/z 609.

### Intermediate 5

### tert-Butyl (2S,2′S)-1,1′-(1S,1′S)-2,2′-((2S,2′S)-2,2′-(1S,1′S,2R,2′R)-2,2′-(hexa-2,4-diyne-1,6-diylbis(oxy))bis(2,3-dihydro-1H-indene-2,1-diyl)bis(azanediyl)bis(oxomethylene)bis(pyrrolidine-2,1-diyl))bis(1-cyclohexyl-2-oxoethane-2,1-diyl)bis(azanediyl)bis(1-oxopropane-2,1-diyl)bis(methylcarbamate)

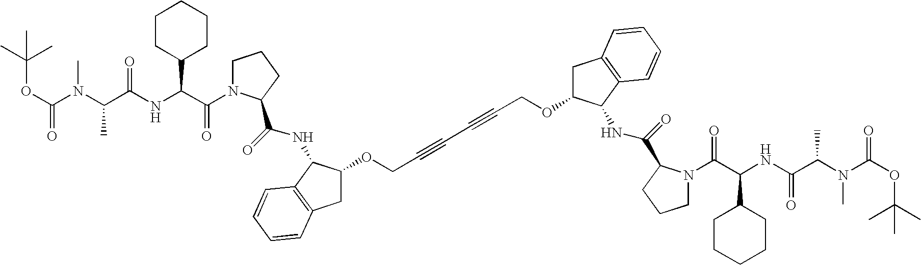

A solution of tert-butyl (S)-1-((S)-1-cyclohexyl-2-oxo-2-((S)-2-((1S,2R)-2-(prop-2-ynyloxy)-2,3-dihydro-1H-inden-1-ylcarbamoyl)pyrrolidin-1-yl)ethylamino)-1-oxopropan-2-yl(methyl)carbamate (Intermediate 4, 340 mg, 0.56 mmol) and copper (II) acetate (505 mg, 2.78 mmol) in MeCN (6.0 mL) was heated to 80° C. for 1 h. The solvents were removed under reduced pressure and H₂O was then added. The resulting mixture was extracted with EtOAc. The solvents were removed under reduced pressure and the residue was purified by an Isco system using a hexanes/EtOAc gradient (silica cartridge) to give the desired product (245 mg, 72%). NMR (DMSO-d6, 100° C.) 7.58-7.66 (m, 2H), 7.15-7.28 (m, 8H), 7.07-7.13 (m, 2H), 5.30-5.37 (m, 2H), 4.50-4.58 (m, 4H), 4.43-4.49 (m, 2H), 4.30-4.40 (m, 6H), 3.70-3.78 (m, 2H), 3.56-3.63 (m, 2H), 2.99-3.10 (m, 4H), 2.74-2.82 (m, 6H), 1.98-2.09 (m, 6H), 1.82-1.94 (m, 2H), 1.56-1.78 (m, 12H), 1.41-1.47 (m, 18H), 1.23-1.26 (m, 6H), 0.91-1.20 (m, 10H); m/z 1216.

### Intermediate 6

### (S)-Methyl 2-(S)-2-(tert-butoxycarbonyl(methyl)amino)propanamido)-2-cyclohexylacetate

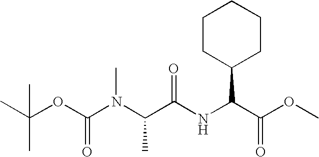

A solution of (S)-methyl-2-amino cyclohexyl acetate hydrochloride (2.07 g, 0.1 mol) and (S)-2-(tert-butoxycarbonyl(methyl)amino)propanoic acid (2.03 g, 0.1 mol) in EtOAc (50 mL) under nitrogen was treated with 2-chloro-4,6-dimethoxy-1,3,5-triazine (CDMT) (1.83 g, 0.11 mol). The reaction mixture was cooled to 0° C. and treated with N-methylmorpholine (2.5 g, 2.5 mol). The reaction mixture was warmed to room temperature and stirred for 1.5 h. The solid precipitate was removed by filtration and rinsed with EtOAc (50 mL). The combined organics were washed with NaHCO₃(sat) and then 10% citric acid. The organics were washed with NaCl(sat) and then removed under reduced pressure to give the desired product (2.7 g, 76% yield) m/z 357.

### Intermediate 7

### (S)-2-(S)-2-(tert-Butoxycarbonyl(methyl)amino)propanamido)-2-cyclohexylacetic acid

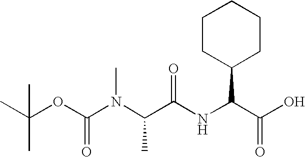

A solution of (S)-Methyl 2-((S)-2-(tert-butoxycarbonyl(methyl)amino)-propanamido)-2-cyclohexylacetate (Intermediate 6, 1.8 g, 5 mmol) in THF (30 mL) was cooled to −5° C. A lithium hydroxide solution (273 mg, 6.5 mmol) was added while keeping the temperature below −17° C. After complete addition, the mixture was warmed to room temperature and stirred for 1 h. The reaction was quenched with 2 M citric acid and extracted with EtOAc. The organic layer was washed with water and then dried with MgSO₄(s). The solvents were removed under reduced pressure to give the desired product; m/z 341 (M-H).

### Intermediate 8

### tert-Butyl (2S,2′S)-1,1′-(1S,1′S)-2,2′-((2S,2′S)-2,2′-(1S,1′S,2R,2′R)-2,2′-(hexane-1,6-diylbis(oxy))bis(2,3-dihydro-1H-indene-2,1-diyl)bis(azanediyl)bis(oxomethylene)bis(pyrrolidine-2,1-diyl))bis(1-cyclohexyl-2-oxoethane-2,1-diyl)bis(azanediyl)bis(1-oxopropane-2,1-diyl)bis(methylcarbamate)

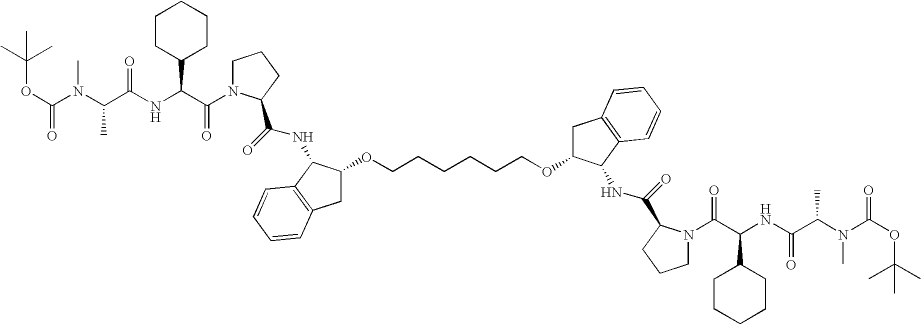

A solution of tert-butyl (2S,2′S)-1,1′-(1S,1′S)-2,2′-((2S,2′S)-2,2′-(1S,1′S,2R,2′R)-2,2′-(hexa-2,4-diyne-1,6-diylbis(oxy))bis(2,3-dihydro-1H-indene-2,1-diyl)bis(azanediyl)bis(oxomethylene)bis(pyrrolidine-2,1-diyl))bis(1-cyclohexyl-2-oxoethane-2,1-diyl)bis(azanediyl)bis(1-oxopropane-2,1-diyl)bis(methylcarbamate) (Intermediate 5, 324 mg, 0.27 mmol) and 10% Pd/C (88 mg) in MeOH (5 mL) was treated with an H₂ atmosphere supplied by a balloon. The reaction mixture was stirred for 4 h at room temperature and then filtered through Celite. The solvents were removed under reduced pressure to give the desired product (284 mg, 87%); m/z 1223.

### Intermediate 9

### (S)-2-(tert-butoxycarbonyl([¹³C, ²H₃]methyl)amino)propanoic acid

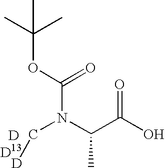

Sodium hydride, 60% dispersion in mineral oil, (1374 mg, 34.35 mmol) was added portionwise (vigorous gas evolution occurred) to a stirred solution of (S)-2-(tert-butoxycarbonylamino)propanoic acid (650 mg, 3.44 mmol) and iodomethane-¹³C-d₃ (5014 mg, 34.35 mmol) in THF (anhydrous, 28 mL) at 0° C. under an atmosphere of nitrogen. The mixture was allowed to warm to room temperature and stirred for 24 hours. Water (7.5 ml) was added followed by EtOAc (5 ml) and the mixture was concentrated under reduced pressure. The residue was diluted with water (150 ml) and washed with EtOAc (75 ml). The aqueous phase was then adjusted to pH 3.5 with citric acid (5% solution in water) and then extracted with EtOAc (100 ml). The organic phase was washed with saturated NaCl (50 ml) and dried with solid MgSO₄ then filtered. The volatiles were removed under reduced pressure to give intermediate 9 (671 mg, 94%). ¹H NMR (CDCl₃, 30° C.) 8.56 (s, 1H), 4.99-4.19 (m, 1H), 1.48-1.42 (m, 9H), 1.42-1.39 (m, 3H); m/z (M-H)⁻206.

### Intermediate 10

### tert-butyl (S)-1-((S)-1-cyclohexyl-2-oxo-2-((S)-2-((1S,2R)-2-(prop-2-ynyloxy)-2,3-dihydro-1H-inden-1-ylcarbamoyl)pyrrolidin-1-yl)ethylamino)-1-oxopropan-2-yl([¹³C, ²H₃]methyl)carbamate

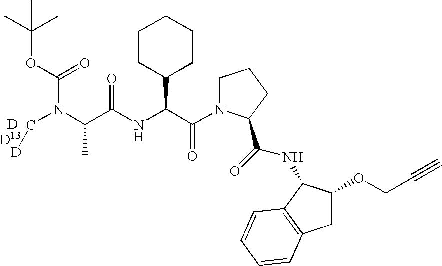

N-((ethylimino)methylene)-N′,N′-dimethylpropane-1,3-diamine hydrochloride (592 mg, 3.09 mmol) was added to a stirred solution of (S)-1-((S)-2-amino-2-cyclohexylacetyl)-N-((1S,2R)-2-(prop-2-ynyloxy)-2,3-dihydro-1H-inden-1-yl)pyrrolidine-2-carboxamide (1100 mg, 2.60 mmol), intermediate 9 (597 mg, 2.88 mmol), 1H-benzo[d][1,2,3]triazol-1-ol (407 mg, 3.01 mmol) and 4-methylmorpholine (337 μl, 3.06 mmol) in DMF (anhydrous, 8650 μl) under an atmosphere of nitrogen. The mixture was allowed to stir at room temperature for 20 hours, and then was partitioned between EtOAc (20 mL) and water (40 mL). The aqueous layer was extracted with EtOAc (three times with 20 mL each time), and the combined organics were washed with 1 N HCl (20 mL), half-saturated NaHCO₃ (20 mL), water (20 mL), and saturated NaCl (20 mL), and were then dried with solid MgSO₄ and filtered. The volatiles were removed under reduced pressure. EtOAc (20 ml) was added and the volatiles were removed under reduced pressure again to give intermediate 10 (1250 mg, 79%). NMR (CDCl₃, 30° C.) 7.24-7.11 (m, 5H), 6.60 (s, 1H), 5.49-5.40 (m, 1H), 4.72-4.56 (m, 2H), 4.56-4.49 (m, 1H), 4.48-4.43 (m, 1H), 4.26-4.16 (m, 2H), 3.84-3.73 (m, 1H), 3.66-3.58 (m, 1H), 3.12-2.98 (m, 2H), 2.47-2.36 (m, 2H), 2.24-2.05 (m, 1H), 2.06-1.86 (m, 2H), 1.63-1.52 (m, 6H), 1.46 (s, 9H), 1.32-1.26 (m, 3H), 1.14-0.81 (m, 5H); m/z 613.

### Intermediate 11

### tert-butyl (2S,2′S)-1,1′-(1S,1′S)-2,2′-((2S,2′S)-2,2′-(1S,1′S,2R,2′R)-2,2′-(hexa-2,4-diyne-1,6-diylbis(oxy))bis(2,3-dihydro-1H-indene-2,1-diyl)bis(azanediyl)bis(oxomethylene)bis(pyrrolidine-2,1-diyl))bis(1-cyclohexyl-2-oxoethane-2,1-diyl)bis(azanediyl)bis(1-oxopropane-2,1-diyl)bis([¹³C, ²H₃]methylcarbamate)

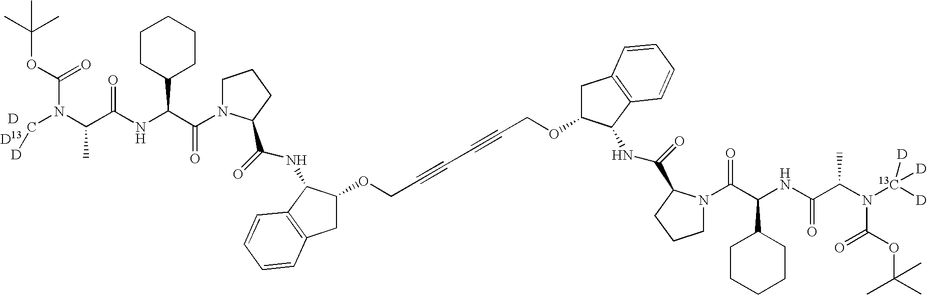

Copper(I) chloride (213 mg, 2.15 mmol) was added to a stirred solution of intermediate 10 (1200 mg, 1.96 mmol) and N,N,N′,N′-tetramethylethane-1,2-diamine (325 μl, 2.15 mmol) in acetone (4571 μl). The resulting suspension was evacuated and flushed with oxygen (four times) then left to stir under an atmosphere of oxygen for 2.5 hours. The mixture was partitioned between EtOAc (60 ml) and water (50 ml) and the aqueous layer further extracted with EtOAc (twice with 50 ml each time). The combined organic layers were washed with aqueous ammonia (2.5 M, 50 ml), water (50 ml) and saturated NaCl (50 ml) and dried (Na₂SO₄). The mixture was filtered and the volatiles were removed under reduced pressure. Diethyl ether (20 ml) was added and the volatiles were removed under reduced pressure again to give intermediate 11 (1213 mg, 100%). NMR (CDCl₃, 30° C.) 7.23-7.11 (m, 10H), 6.59 (s, 2H), 5.49-5.38 (m, 2H), 4.76-4.55 (m, 4H), 4.55-4.48 (m, 2H), 4.48-4.39 (m, 2H), 4.35-4.23 (m, 4H), 3.86-3.73 (m, 2H), 3.68-3.57 (m, 2H), 3.09-3.01 (m, 4H), 2.48-2.35 (m, 2H), 2.24-2.06 (m, 2H), 2.01-1.84 (m, 4H), 1.68-1.56 (m, 12H), 1.46 (s, 18H), 1.28 (d, J=7.1 Hz, 6H), 1.10-0.83 (m, 10H); m/z 1224.

### Intermediate 12

### tert-butyl (2S)-2-[[(1S,2R)-2-hydroxyindan-1-yl]carbamoyl]pyrrolidine-1-carboxylate

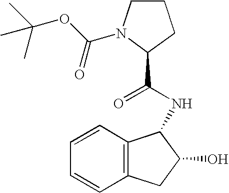

Boc-L-Proline (1228 g, 5.70 moles) was slurried in EtOAc (13 L) and cooled to 0° C. 4-Methylmorpholine (659 ml, 5.99 moles) was added and the resulting solution stirred at 0° C. for 10 minutes. Ethyl chloroformate (570 ml, 5.97 moles) was added over 1 hour maintaining the temperature below 5° C. The reaction mixture was then stirred at 0-5° C. for 30 minutes. 1S,2R-1-Amino-2-indanol (885 g, 5.93 moles) was added over 30 minutes and the mixture allowed to warm to room temperature overnight. The reaction mixture was washed with 1M HCl (twice with six liters each time). The aqueous was extracted with EtOAc (5.5 L). The combined organic extracts were washed with half saturated NaHCO₃ (8 L) and saturated NaCl (5.5 L) and dried over sodium sulfate (300 g). The volatiles were removed under reduced pressure to leave a white solid which was slurried in EtOAc (7 L) at room temperature for 30 minutes. Heptane (12 L) was added and the mixture stirred for 1 hour at 0 to 5° C. The solid was collected by filtration and the filter cake washed with heptane (twice with one liter each time). The filter cake was air dried to give intermediate 12 as a white solid in 83% yield (1642 g) with a purity of 100% by HPLC (>95% by 1H NMR). NMR: (DMSO-d6, 100° C.)) 7.30-7.37 (m., 1H), 7.13-7.23 (m, 4H), 5.18 (dd, 1H), 4.62 (d, 1H), 4.38-4.51 (m, 1H), 4.28 (dd, 1H), 3.38 (dd, 2H), 3.08 (dd, 1H), 2.83-2.92 (m, 1H), 1.96-2.19 (m, 2H), 1.72-1.96 (m, 2H), 1.41 (s, 9H). m/z 346.

### Intermediate 13

### tert-butyl (2S)-2-[[(1S,2R)-2-prop-2-ynoxyindan-1-yl]carbamoyl]pyrrolidine-1-carboxylate

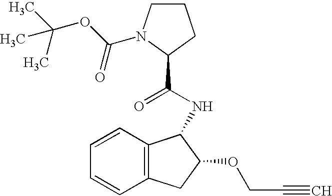

Intermediate 12 (1632 g, 4.71 moles) was dissolved in anhydrous DMF (10 L) and cooled to below 5° C. Propargyl bromide (606 ml of an 80% solution in toluene, 5.63 moles) was added over 10 minutes. Freshly ground KOH (541.3 g, 8.20 moles) was added over 10 minutes. The resulting slurry was stirred at 0 to 5° C. for 2 hours. EtOAc (5 L) was added followed by water (5 L). Water (6 L) and EtOAc (11 L) were added and the layers separated. The layer was extracted with EtOAc (10 L). The organic extracts were washed with water (five times with two liters each time) and saturated NaCl (three times with three liters each time) then dried over solid Na₂SO₄ (500 g) and filtered. The volatiles were removed under reduced pressure to leave a yellow oil which solidified on standing. The crude product was dissolved in EtOAc (2.7 L) then heptane (11 L) was added. The mixture was cooled in an ice bath and a precipitate slowly formed. After stirring for 1 hour, heptane (4 L) was added and the solid collected by filtration. The solid was washed with heptane (4 L) and dried under reduced pressure at 30° C. to give intermediate 13 in 74% yield (1349 g corrected for solvent content—contained 23% heptane) with a purity of 94.7% by HPLC. 1H NMR (400 MHz, DMSO-d6, 100° C.) 7.46-7.48 (m, 1H) 7.16-7.29 (m, 4H) 5.34 (dd, 1H) 4.35-4.45 (m, 1H) 4.27-4.30 (m, 1H) 4.12-4.25 (m, 2H) 3.30-3.46 (m, 2H) 3.17 (t, 1H) 3.06 (d, 2H) 2.06-2.20 (m, 1H) 1.94-2.06 (m, 1H) 1.85-1.94 (m, 1H) 1.73-1.85 (m, 1H) 1.40 (s, 9H). m/z 385.

### Intermediate 14

### tert-butyl N-[1-cyclohexyl-2-oxo-2-[2-[(2-prop-2-ynoxyindan-1-yl)carbamoyl]pyrrolidin-1-yl]ethyl]carbamate

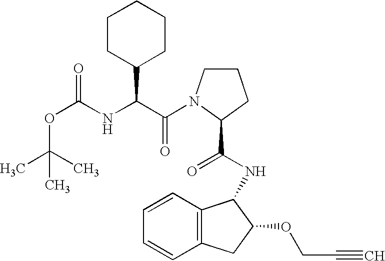

The material from intermediate 3, alternate 2 (1180 g, 2.96 moles), Boc-cyclohexylglycine (801 g, 3.11 moles) and HOBt hydrate (530 g, 3.46 moles) were dissolved in anhydrous DMF (9.9 L) and cooled to 0° C. N-Methylmorpholine (365 ml, 3.34 moles) was added over 30 minutes and the mixture stirred at 0 to 5° C. for 30 minutes. 1-ethyl-3-(3-dimethylaminopropyl)carbodiimide (EDCI) (636 g, 3.32 moles) was added followed by anhydrous DMF (1.1 L). The reaction mixture was allowed to warm to room temperature and stirred overnight. EtOAc (7.4 L) was added and the solution was washed with water (18 L). The aqueous layers were extracted with EtOAc (twice with 7.4 liters each time). The organic extracts were washed with 1M HCl (7.8 L), half saturated sodium bicarbonate solution (6.8 L), water (twice with 6.7 L each time) and saturated NaCl (3.4 L). The organic extracts were then dried over sodium sulfate (1 Kg) and the volatiles were removed under reduced pressure to give an oil (1653 g, 91% purity by HPLC). The crude product was purified by column chromatography on silica (2×10 Kg) eluting with 1% methanol in DCM. The volatiles of the relevant fractions were removed under reduced pressure and gave intermediate 14 as a white foam (1282 g, 83%) with a purity of 98.7% by HPLC. m/z 524.

### Intermediate 15

### (2S)-1-[(2S)-2-amino-2-cyclohexyl-acetyl]-N-[(1S,2R)-2-prop-2-ynoxyindan-1-yl]pyrrolidine-2-carboxamide

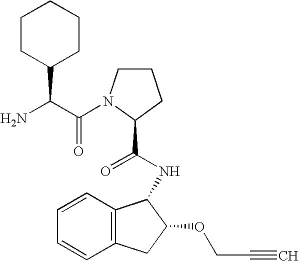

To a solution of intermediate 14 (1273 g, 2.43 moles) in DCM (3.14 L) was added TFA (1.564 L, 21.05 moles) dropwise over 30 minutes. The reaction was then stirred at ambient temperature overnight. The reaction mixture was concentrated under reduced pressure and the residue was redissolved in DCM (5.46 L) and washed with saturated NaHCO₃ (2.73 L). The aqueous layer was extracted with DCM (twice with 2.73 L each time). The combined organic layers were washed with saturated NaHCO₃ (2.73 L) and dried with Na₂SO₄. The volatiles were removed under reduced pressure to afford 1050 g of intermediate 15 as a yellow gum (about 20% DCM, 826 g active content). HPLC analysis indicated the product had a purity of greater than 98.8%. m/z 424.

### Intermediate 16

### tert-butyl N-[(1S)-2-[[(1S)-1-cyclohexyl-2-oxo-2-[(2S)-2-[[(1S,2R)-2-prop-2-ynoxyindan-1-yl]carbamoyl]pyrrolidin-1-yl]ethyl]amino]-1-methyl-2-oxo-ethyl]-N-methyl-carbamate

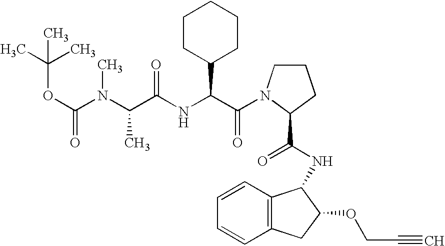

Intermediate 15 (755 g, 1.78 moles), Boc-NMe-Ala-OH (465 g, 2.29 moles) and HOBt hydrate (382 g, 2.50 moles) were dissolved in anhydrous DMF (5570 ml) and cooled to 5° C. N-Methylmorpholine (260 ml, 2.44 moles) was added over 15 minutes at less than 5° C. Anhydrous DMF (650 ml) was added. The mixture was stirred at 0 to 5° C. for 15 minutes. EDCI (466 g, 2.43 moles) was added over 15 minutes at less than 5° C. Anhydrous DMF (650 ml) was added. The reaction mixture was allowed to warm to room temperature and stirred overnight. EtOAc (9250 ml) was added and washed with water (21 L). The aqueous layers were then extracted with EtOAc (three times with nine liters each time). The combined organic extracts were washed with 1M HCl (twice with 11 liters each time), saturated NaHCO₃ (twice with 4620 ml each time), water (three times with 5.3 liters each time) and saturated NaCl (4.6 L). The combined organic extracts were dried over sodium sulphate (200 g) and the volatiles were removed under reduced pressure to leave a yellow gum. DCM (twice with one liter each time and twice with five liters each time) was added and the volatiles were removed under reduced pressure under reduced pressure. Intermediate 16 was isolated as a yellow gum in 97% yield (1051 g) with a purity of 94.0% by HPLC and 99% by 1H NMR assay (corrected for solvent content). m/z 609.

### Intermediate 17

### tert-butyl N-[(1S)-2-[[(1S)-2-[(2S)-2-[[(1S,2R)-2-[6-[(1S,2R)-1-[[(2S)-1-[(2S)-2-[[(2S)-2-[tert-butoxycarbonyl(methyl)amino]propanoyl]amino]-2-cyclohexyl-acetyl]pyrrolidine-2-carbonyl]amino]indan-2-yl]oxyhexa-2,4-diynoxy]indan-1-yl]carbamoyl]pyrrolidin-1-yl]-1-cyclohexyl-2-oxo-ethyl]amino]-1-methyl-2-oxo-ethyl]-N-methyl-carbamate

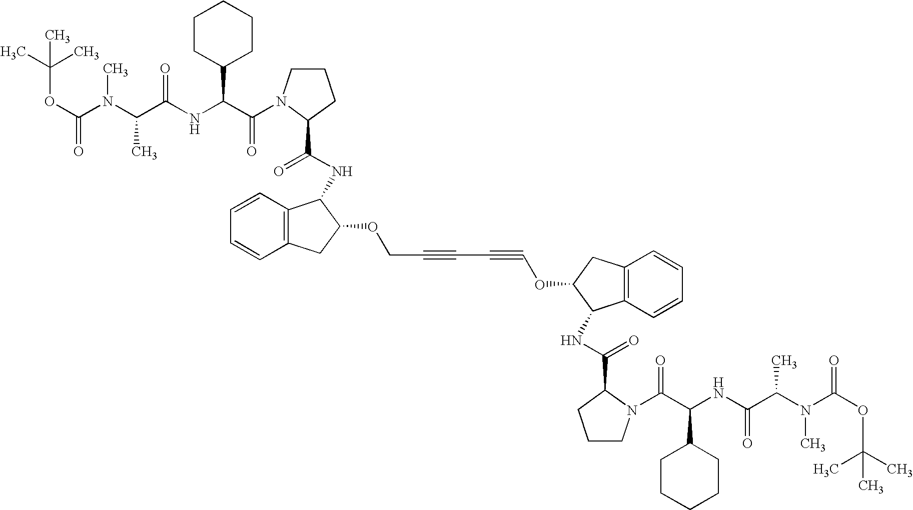

Under an air atmosphere, CuCl (179.96 g, 1.81 moles) was added to a solution of intermediate 16 (1.00 Kg, 1.64 moles) in DCM (1.94 L), followed by DCM (3 L) and tetramethylethylenediamine (1.97 moles, 297 mL). After an overnight hold at 20° C., water (3.00 L) was added, and the upper aqueous portion was separated off 10% ammonia (3.00 L) was added, and the upper aqueous portion was separated off 10% ammonia (3.00 L) was added, and the upper aqueous portion was separated off. The solvents were removed under reduced pressure, and 1090 g of intermediate 17 was obtained (78% w/w, 85.1% yield). m/z 1215. Intermediate 17 was purified by Hipersep® chromatography using 3% MeOH in methyl-tert-butyl ether (MTBE) as eluent. 837 g in 404 L were obtained, and the solvents removed under reduced pressure.

### Intermediate 18

### (S)-tert-butyl 2-((1S,2 S)-2-hydroxy-2,3-dihydro-1H-inden-1-ylcarbamoyl)pyrrolidine-1-carboxylate

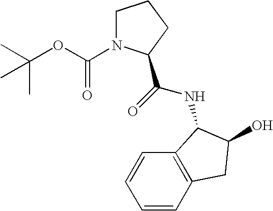

A mixture of (S)-1-(tert-butoxycarbonyl)pyrrolidine-2-carboxylic acid (2.89 g, 13.41 mmol) in EtOAc (40 ml) was cooled to 0° C. via ice-water bath. 4-methylmorpholine (1.474 ml, 13.41 mmol) was added to the mixture followed by the dropwise addition of ethyl carbonochloridate (1.282 ml, 13.41 mmol). After the reaction mixture was stirred at 0° C. for 30 min, (1S,2S)-1-amino-2,3-dihydro-1H-inden-2-ol (2.0 g, 13.41 mmol) was added. The resulting mixture was then allowed to warm to room temperature overnight with stirring. The reaction mixture was diluted with water and extracted with EtOAc. The organics were then washed with 10% citric acid, saturated NaCl, and aqueous NaHCO₃. The organics were dried over solid MgSO₄, and the volatiles were removed under reduced pressure to give intermediate 18 (4.59 g, 99%). NMR (400 MHz, DMSO-d₆) 8.14-8.23 (m, 1H), 7.10-7.22 (m, 4H), 5.15-5.26 (m, 1H), 4.92-5.04 (m, 1H), 4.26-4.35 (m, 1H), 4.23 (m, 1H), 4.13 (m, 1H), 3.35-3.43 (m, 1H), 3.06-3.18 (m, 1H), 2.69 (m, 1H), 2.05-2.17 (m, 1H), 1.71-1.97 (m, 3H), 1.32-1.44 (m, 9H); m/z 347.

### Intermediate 19

### (S)-tert-butyl 2-((1S,2S)-2-(prop-2-ynyloxy)-2,3-dihydro-1H-inden-1-ylcarbamoyl)pyrrolidine-1-carboxylate

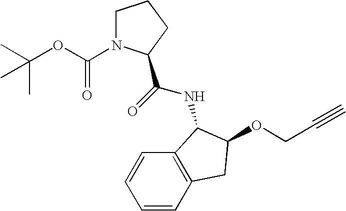

A solution of intermediate 18 (2.49 g, 7.19 mmol) in DMF (16.81 ml) was cooled to 0° C. 3-bromoprop-1-yne (80 weight percent in toluene, 1.162 ml, 10.78 mmol) was added to the solution followed by powdered KOH (0.807 g, 14.38 mmol). The resulting mixture was stirred for 60 min at 0° C. The mixture was then diluted with EtOAc and water. The aqueous layer was extracted with EtOAc. The combined organics were washed with saturated NaCl and dried over solid MgSO₄. The volatiles were removed under reduced pressure and the product was purified by an Isco system (0-100% EtOAc in hexanes gradient) to give intermediate 19 (1.34 g, 49%); m/z 385.

### Intermediate 20

### (S)—N-((1S,2S)-2-(prop-2-ynyloxy)-2,3-dihydro-1H-inden-1-yl)pyrrolidine-2-carboxamide

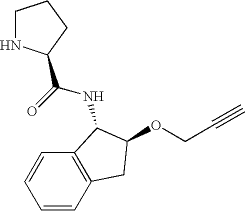

HCl (4 M in Dioxane, 30 ml, 120 mmol) was added to intermediate 19 (1.34 g, 3.49 mmol). The reaction solution was stirred at room temperature for 30 min. The volatiles were then removed under reduced pressure and placed under reduced pressure for 48 h. The product was used directly in the next step.

### Intermediate 21

### tert-butyl (S)-1-((S)-1-cyclohexyl-2-oxo-2-((S)-2-((1S,2S)-2-(prop-2-ynyloxy)-2,3-dihydro-1H-inden-1-ylcarbamoyl)pyrrolidin-1-yl)ethylamino)-1-oxopropan-2-yl(methyl)carbamate

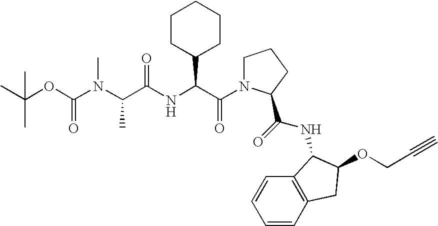

A mixture of intermediate 20 (0.992 g, 3.49 mmol), intermediate 8 (1.195 g, 3.49 mmol) in EtOAc (23 mL) and 4-methylmorpholine (0.384 mL, 3.49 mmol) was cooled to −20° C. 4-(4,6-Dimethoxy[1.3.5]triazin-2-yl)-4-methylmorpholinium chloride hydrate (1.024 g, 3.70 mmol) was added to the mixture at −20° C. and stirred for 10 min. The mixture was then allowed to warm to room temperature and stirred overnight. The mixture was filtered and washed with EtOAc. The combined filtrate was washed with 10% citric acid and saturated NaHCO₃, saturated NaCl and dried with solid MgSO₄. The volatiles were removed under reduced pressure. The product was purified using an ISCO system (0-100% EtOAc in hexanes) to give intermediate 21 (1.44 g, 68%); m/z 609.

### Intermediate 22

### tert-butyl (2S,2′S)-1,1′-(1S,1′S)-2,2′-((2S,2′S)-2,2′-(1S,1′S,2S,2′S)-2,2′-(hexa-2,4-diyne-1,6-diylbis(oxy))bis(2,3-dihydro-1H-indene-2,1-diyl)bis(azanediyl)bis(oxomethylene)bis(pyrrolidine-2,1-diyl))bis(1-cyclohexyl-2-oxo ethane-2,1-diyl)bis(azanediyl)bis(1-oxopropane-2,1-diyl)bis(methylcarbamate)

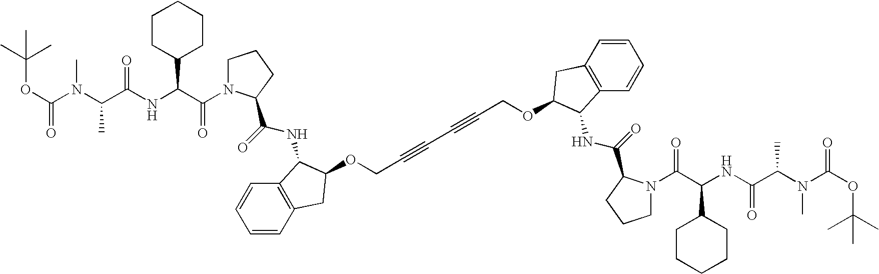

A mixture of intermediate 21 (0.50 g, 0.82 mmol), cupric acetate (0.224 g, 1.23 mmol) and pyridine (0.266 ml, 3.29 mmol) in acetonitrile (7.95 ml) was heated at 80° C. for 2 h. The mixture was then diluted with EtOAc and washed with water. The aqueous layer was further extracted with EtOAc. The combined organics were dried, and the volatiles were removed under reduced pressure. The crude product was purified by an ISCO system (0-10% MeOH in EtOAc), and then purified by a Gilson HPLC (80-95% MeCN in water with 0.1% TFA over 14 min) to give 341 mg of the product. A second Gilson HPLC purification (75-90% MeCN in H₂O with 0.1% TFA over 15 min) gave intermediate 22 (0.117 g, 23%); m/z 1216.

### Examples

### Example 1a

tert-Butyl (2S,2′S)-1,1′-(1S,1′S)-2,2′-((2S,2′S)-2,2′-(1S,1′S,2R,2′R)-2,2′-(hexa-2,4-diyne-1,6-diylbis(oxy))bis(2,3-dihydro-1H-indene-2,1-diyl)bis(azanediyl)bis(oxomethylene)bis(pyrrolidine-2,1-diyl))bis(1-cyclohexyl-2-oxoethane-2,1-diyl)bis(azanediyl)bis(1-oxopropane-2,1-diyl)bis(methylcarbamate) (Intermediate 5, 230 mg, 0.19 mmol) was treated with 4 N HCl/dioxane (5.0 mL), and the resulting solution was stirred at room temperature for 1 h. The solvents were removed under reduced pressure. The crude material was purified by a Gilson HPLC (30-75% MeCN/0.1% TFA in H₂O/0.1% TFA over 14 minutes). The combined fractions were partitioned between EtOAc and saturated NaHCO₃. The combined organics were washed with NaCl(sat) and then dried with Na₂SO₄(s). The solvents were removed under reduced pressure. A solution of MeCN/H₂O was added and then lyophilized to give compound 1. NMR (DMSO-d6, 100° C.) 7.55-7.66 (m, 4H), 7.16-7.28 (m, 8H), 5.32-5.38 (m, 2H), 4.52-4.58 (m, 2H), 4.44-4.51 (m, 2H), 4.31-4.40 (m, 6H), 3.72-3.82 (m, 2H), 3.55-3.64 (m, 2H), 3.03-3.07 (m, 4H), 2.97-3.01 (m, 2H), 2.25 (s, 6H), 1.97-2.08 (m, 6H), 1.85-1.93 (m, 2H), 1.56-1.78 (m, 12H), 0.97-1.23 (m, 16H); m/z 1016.

### Example 1b

A solution of intermediate 17 (12.37 g, 10.18 mmoles) and p-toluenesulfonic acid hydrate (4.26 g, 22.39 mmoles) in EtOH (61.85 mL) were heated to 70° C. for 4 hours. The solution was cooled down and added to MTBE (125 mL), causing compound 1 to precipitate out of solution as a bis 4-methylbenzenesulfonic acid (tosylate) salt. The compound 1 bis tosylate salt was filtered under reduced pressure and washed with MTBE (37 mL) and further dried in vacuo to constant weight (11.70 g, 97% w/w, 82% yield). m/z 1015.

### Example 1c

A ¹³CD₃ labelled version of compound 1 was made as follows. HCl (4M) in dioxane (2452 μl, 9.81 mmol) was added to a stirred solution of intermediate 11 (1200 mg, 0.98 mmol) in methanol (712 μl) under an atmosphere of nitrogen for 2 hours. The mixture was concentrated, methanol (10 ml) was added to the sticky residue and the mixture was again concentrated. The residue was partitioned between ethyl acetate (20 ml) and sodium bicarbonate (20 ml, saturated aqueous) and stirred vigorously for 30 minutes. The organic phase was removed and the aqueous phase was extracted with more ethyl acetate (twice with 20 ml each time) and the combined organic phases were washed with water (20 ml), dried with Na₂SO₄ and filtered. The mixture was evaporated to dryness and dissolved in isopropanol (7 ml). Benzoic acid (240 mg, 1.96 mmol) was added and the mixture heated to reflux to give a clear solution. The mixture was then allowed to cool to room temperature, the precipitate was collected by filtration and washed with more IPA (7 ml) and dried under reduced pressure at room temperature to give compound 1c (941 mg, 0.742 mmol, 76%). NMR (DMSO, 100° C.) 7.99-7.91 (m, 4H), 7.68-7.51 (m, 6H), 7.51-7.45 (m, 4H), 7.28-7.14 (m, 8H), 5.40-5.28 (m, 2H), 4.61-4.51 (m, 2H), 4.51-4.42 (m, 2H), 4.40-4.27 (m, 6H), 3.84-3.69 (m, 2H), 3.65-3.55 (m, 2H), 3.12-2.94 (m, 8H), 2.12-1.96 (m, 6H), 1.94-1.80 (m, 2H), 1.79-1.55 (m, 12H), 1.24-0.94 (m, 16H); m/z 1024.

### Example 2

The following compound was prepared by the procedure of Example 1, using the indicated starting materials.

| | | | Starting |
|---|---|---|---|
| Ex. | NMR (DMSO-d6) | m/z | material |
| 2 | (100° C.) 7.55-7.66 (m, 2 H), 7.16-7.26 | 510 | Interme- |
| | (m, 4 H), 5.30-5.35 (m, 1 H), 4.53-4.58 (m, 1 H), | | diate 4 |
| | 4.45-4.51 (m, 1 H), 4.35-4.42 (m, 1 H), 4.15-4.23 | | |
| | (m, 2 H), 3.74-3.80 (m, 1 H), 3.57-3.65 (m, 1 H), | | |
| | 3.16-3.19, (m, 1 H), 2.96-3.09 (m, 3 H) 2.26 | | |
| | (s, 3 H), 1.99-2.09 (m, 3 H), 1.85-1.93 (m, 1 H), | | |
| | 1.58-1.79 (m, 6 H), 0.95-1.25 (m, 8 H) | | |

### Example 3

tert-Butyl (2S,2′S)-1,1′-(1S,1′S)-2,2′-((2S,2′S)-2,2′-(1S,1′S,2R,2′R)-2,2′-(hexane-1,6-diylbis(oxy))bis(2,3-dihydro-1H-indene-2,1-diyl)bis(azanediyl)bis(oxomethylene)bis(pyrrolidine-2,1-diyl))bis(1-cyclohexyl-2-oxoethane-2,1-diyl)bis(azanediyl)bis(1-oxopropane-2,1-diyl)bis(methylcarbamate) (Intermediate 8, 284 mg, 0.23 mmol) was treated with 4 N HCl/dioxane (6.0 mL), and the resulting mixture was stirred at room temperature for 1 h. The solvents were then removed under reduced pressure and the residue was dissolved in MeOH and the resulting solution was filtered through a plug of Celite® (diatomaceous earth). The solvents were removed under reduced pressure, and the residue was triturated with ether. Finally, removal of the ether under reduced pressure afforded compound 3 (254 mg, 100%). NMR (DMSO-d6, 100° C.) 7.50-7.55 (m, 2H), 7.14-7.26 (m, 8H), 5.28-5.32 (m, 2H), 4.54-4.60 (m, 2H), 4.45-4.49 (m, 2H), 4.17-4.21 (m, 2H), 3.85-3.94 (m, 2H), 3.70-3.79 (m, 2H), 3.59-3.64 (m, 2H), 3.43-3.47 (m, 4H), 2.90-3.07 (m, 4H), 2.51 (m, 6H), 1.96-2.09 (m, 6H), 1.84-1.93 (m, 2H), 1.58-1.83 (m, 12H), 1.47-1.55 (m, 4H), 1.38-1.40 (m, 6H), 1.28-1.33 (m, 4H), 1.04-1.23 (m, 10H); m/z=1024.

### Example 4

HCl (4M in Dioxane, 10.0 ml, 40.00 mmol) was added to intermediate 22 (0.117 g, 0.10 mmol). The reaction solution was stirred at room temperature for 1 h. After the solution was concentrated, the residue was diluted with EtOAc and washed with saturated NaHCO₃. The aqueous layer was extracted with EtOAc twice. The combined organic layers were dried with solid MgSO₄, and the volatiles were removed under reduced pressure to give compound 4 (0.040 g, 41%). NMR (DMSO-d₆) 7.96 (bs, 2H), 7.60 (bs, 2H), 7.13-7.25 (m, 8H), 5.16 (m, 2H), 4.38-4.50 (m, 8H), 4.23-4.33 (m, 2H), 3.74-3.81 (m, 2H), 3.66-3.74 (m, 2H), 3.59-3.65 (m, 2H), 3.54 (m, 2H), 3.30 (m, 2H), 3.01 (m, 2H), 2.83 (m, 2H), 2.25 (s, 6H), 1.84-2.15 (m, 8H), 1.58-1.81 (m, 12H), 0.98-1.30 (m, 16H); m/z 1016.

### Biological Assay: Fluorescence Polarization Assay

Materials: The cIAP1 Bir3 domain construct (aa L250-G350) was prepared from a full length cIAP1 clone (NCBI Reference Sequence: NM_{—}001166.3). PCR was used to generate the Bir3 fragment which was inserted into a pGEX-6P-1 vector (GE LifeSciences) as a BamHI/XhoI fragment. Protein was prepared in Escherichia coli BL21 (DE3) grown at 37° C. containing ampicillin to an OD600 of 0.6. Protein expression was induced by 1 mM isopropyl β-D-1-thiogalactopyranoside (IPTG) for 3.75 hours.

Cells were lysed by sonication in buffer containing 100 mM Tris (pH 7.5), 150 mM NaCl, 5 mm dithiothreitol (DTT), 50 μM zinc acetate (ZnAc), ethylenediamine tetraacetic acid (EDTA) free Complete protease inhibitors (Roche Applied Science), 100 μg/mL lysozyme, and 0.5% Triton-X100. DNAse was added to the mixture after sonication. Proteins were purified from the soluble fraction using Glutathione Sepharose 4B resin (GE Lifesciences) followed by desalting on a PD-10 column (GE Lifesciences). This was followed by purification over an SEC 200 resin (GE Lifesciences). Final storage buffer consisted of 25 mM Tris, pH 7.5, 200 mM NaCl, 5 mM DTT, 50 uM ZnAc, 10% glycerol.

The fluorescence polarization assay was developed using Glutathione-S-transferase tagged Bir3 domain of cIAP1 (L250-G350). The tracer used was a synthetic peptide conjugated to 5-carboxyfluorescein (AbuRPF-K-5FAM).

Methods: Dilutions of cIAP1-Bir3 (20 nM) were added to 2.5 nM of fluorescent peptide tracer in assay buffer (final concentrations of 20 mM HEPES, pH 7.5, 1 mM DTT, 0.005% Tween-20 and 50 mM NaCl) containing various dilutions of binding inhibitors. Samples were read by a Tecan Ultra Evolution (Tecan US Inc, Durham N.C.) after a 20-minute incubation.

Fluorescence polarization values were plotted as a function of the antagonist concentration and the IC50 values were determined.

| Example | cIAP1 (microM) |
|---|---|
| 1 | 0.012 |
| 2 | 0.015 |
| 3 | 0.013 |
| 4 | 0.0429 |

Any embodiment described herein can be combined with any other suitable embodiment described herein to provide additional embodiments.

As used herein, reference to “a” or “an” means “one or more.” Throughout, the plural and singular should be treated as interchangeable, other than the indication of number.

As will be understood by one skilled in the art, for any and all purposes, particularly in terms of providing a written description, all ranges disclosed herein also encompass any and all possible subranges and combinations of subranges thereof as well as the individual values making up the range, particularly integer values. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. For example, the range C(1-6), includes the subranges C(2-6), C(3-6), C(3-5), C(4-6), etc., as well as C1 (methyl), C2 (ethyl), C3 (propyl), C4 (butyl), C5 (pentyl) and C6 (hexyl) individually. As will also be understood by one skilled in the art, all language such as “up to,” “at least,” “greater than,” “less than,” “more than,” “or more” and the like include the number recited and refer to ranges which can be subsequently broken down into subranges as discussed above. In the same manner, all ratios disclosed herein also include all subratios falling within the broader ratio.

One skilled in the art will also readily recognize that where members are grouped together in a common manner, such as in a Markush group, the present invention encompasses not only the entire group listed as a whole, but each member of the group individually and all possible subgroups of the main group. Additionally, for all purposes, the present invention encompasses not only the main group, but also the main group absent one or more of the group members. The present invention also envisages the explicit exclusion or disclaimer of one or more of any of the group members in the claimed invention.

As will be understood by the skilled artisan, all numbers, including those expressing quantities of ingredients, properties such as molecular weight, reaction conditions, and so forth, are approximations and understood as being modified in all instances by the term “about.” These values can vary depending upon the desired properties sought to be obtained by those skilled in the art utilizing the present teachings of the present invention. It is also understood that such values inherently contain variability necessarily resulting from the standard deviations found in their respective testing measurements.

Reference to a “step” in the application is used for convenience purposes only and does not categorize, define or limit the invention as set forth herein.

While specific embodiments have been described above with reference to the disclosed embodiments and examples, these embodiments and examples are only illustrative and do not limit the scope of the invention. Changes and modifications can be made in accordance with ordinary skill in the art without departing from the invention in its broader aspects as defined in the following claims.

What is claimed is:

1. A compound of formula I: 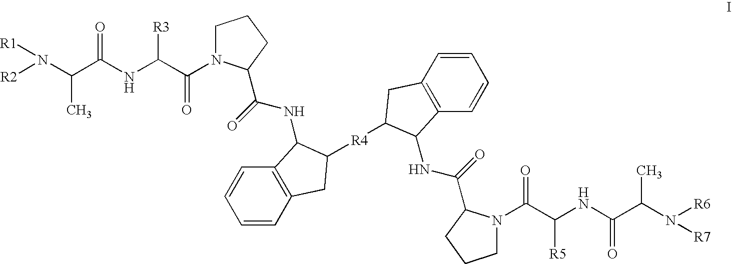
wherein:
R1 and R2 are independently H or C(1-6)alkyl;
R3 is H or a C(3-8)cycloalkyl;
R4 is —OC(3-10)alkylO—, —OC(3-10)alkenylO— or —OC(3-10)alkynylO—,
R5 is H or C(3-8)cycloalkyl; and
R6 and R7 are independently H or C(1-6)alkyl; or
a salt thereof.

2. A compound of claim 1 or a salt thereof wherein one of R1 and R2 is a C(1-6)alkyl and the other of R1 and R2 is H.

3. A compound of claim 2 or a salt thereof wherein one of R1 and R2 is a methyl and the other of R1 and R2 is H.

4. A compound of claim 1 or a salt thereof wherein R3 is cyclohexyl.

5. A compound of claim 1 or a salt thereof wherein R4 is,

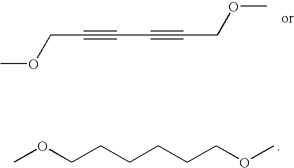

6. A compound of claim 1 or a salt thereof wherein R5 is cyclohexyl.

7. A compound of claim 1 or a salt thereof wherein one of R6 and R7 is a C(1-6)alkyl and the other of R6 and R7 is H.

8. A compound of claim 7 or a salt thereof wherein one of R6 and R7 is a methyl and the other of R6 and R7 is H.

9. A compound of claim for a salt thereof wherein:
R1, R2, and R3 are the same as R6, R7, and R5, respectively.

10. A compound of claim 1 or a salt thereof wherein the compound of Formula I has the structure:

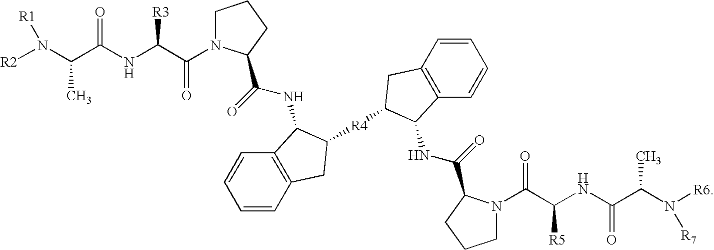

11. A compound of claim 1 selected from: 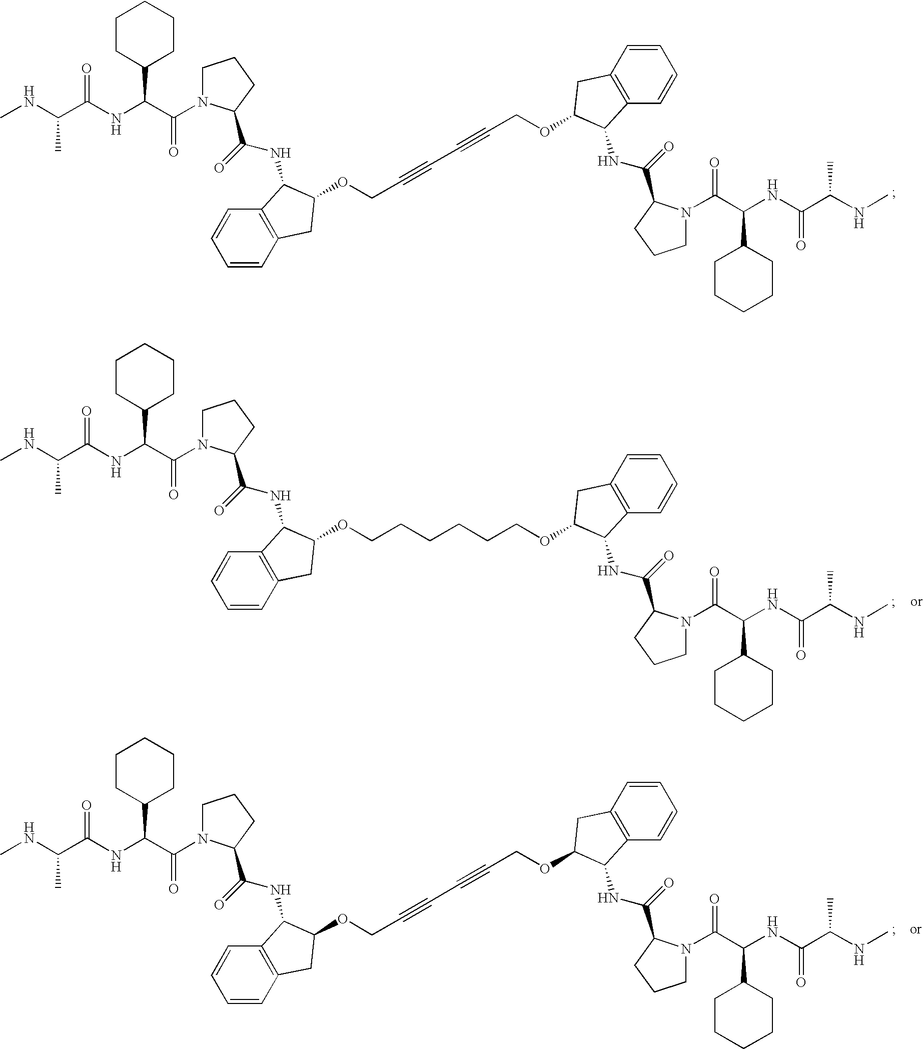
or
a salt thereof.

12. A pharmaceutically acceptable salt of a compound of claim 11.

13. A pharmaceutical composition comprising:
(i) a compound of claim 1 or a pharmaceutically acceptable salt thereof; and
(ii) a pharmaceutically acceptable carrier, diluent or excipient.

14. A method of treating a cancer in a mammal comprising administering an effective amount a compound of claim 1 or a pharmaceutically acceptable salt thereof to the mammal.

15. The method of treating a cancer of claim 15 wherein the cancer is selected from the group consisting of acute myeloid leukemia, bladder cancer, breast cancer, colon cancer, diffuse large B-cell lymphoma, non-small cell lung cancer, ovarian cancer, pancreatic cancer and prostate cancer.
